(19) United States
(12) Patent Application Publication
(10) Pub. No.: US 2023/0213535 A1
(43) Pub. Date: Jul. 6, 2023

(54) PROTEIN MARKERS FOR ASSESSING ALZHEIMER'S DISEASE

(71) Applicant: The Hong Kong University of Science and Technology, Kowloon (CN)

(72) Inventors: Nancy Yuk-Yu IP, Hong Kong SAR (CN); Kit Yu Fu, Kowloon, Hong Kong SAR (CN); Yuanbing Jiang, Chengdu, Sichuan (CN); Xiaopu Zhou, Chongqing (CN); Fanny Chui-Fun Ip, Kowloon (CN)

(21) Appl. No.: 17/996,498

(22) PCT Filed: May 12, 2021
(86) PCT No.: PCT/CN2021/093274

§ 371 Date: Oct. 18, 2022

Related U.S. Application Data
(60) Provisional application No. 63/024,940, filed on May 14, 2020.

(51) Int. Cl. G01N 33/68; C12Q 1/6883

(52) U.S. Cl. CPC G01N 33/6896; C12Q 1/6883; C12Q 1/6869

(57) ABSTRACT

The present invention provides protein markers present in a person's blood sample (such as a plasma, serum, or whole blood sample) that are associated with the Alzheimer's Disease (AD), diagnostic and treatment methods for AD, and kits for diagnosing AD.

### RELATED APPLICATIONS

This application claims priority to U.S. Provisional Patent Application No. 63/024,940, filed May 14, 2020, the contents of which are hereby incorporated by reference in the entirety for all purposes.

### BACKGROUND OF THE INVENTION

Brain diseases such as neurodegenerative diseases and neuroinflammatory disorders are devastating conditions that affect a large subset of the population. Many are incurable, highly debilitating, and often result in progressive deterioration of brain structure and function over time. Disease prevalence is also increasing rapidly due to growing aging populations worldwide, since the elderly are at high risk for developing these conditions. Currently, many neurodegenerative diseases and neuroinflammatory disorders are difficult to diagnose due to limited understanding of the pathophysiology of these diseases. Meanwhile, current treatments are ineffective and do not meet market demand; demand that is significantly increasing each year due to aging populations. For example, Alzheimer's disease (AD) is marked by gradual but progressive decline in learning and memory, and a leading cause of mortality in the elderly. Increasing prevalence of AD is driving the need and demand for better diagnostics. According to Alzheimer's Disease International, the disease currently affects 46.8 million people globally, but the number of cases is projected to triple in the coming three decades. One of the countries with the fastest elderly population growth is China. Based on population projections, by 2030 one in four individuals will be over the age of 60, which will place a vast proportion at risk of developing AD. In fact, the number of AD cases in China doubled from 3.7 million to 9.2 million from 1990-2010, and the country is projected to have 22.5 million cases by 2050. Hong Kong's population is also aging quickly. It is estimated that the elderly aged 65+ will make up 24% of the population by 2025, and 39.3% of the population by 2050. The number of AD cases is projected to rise to 332,688 by 2039.

More worrisome is that, despite the increase in AD prevalence, many people fail to receive a correct AD diagnosis. According to Alzheimer's Disease International's World Alzheimer' Report 2015, in high-income countries only 20-50% of dementia cases are documented in primary care. The rest remain undiagnosed or incorrectly diagnosed. This ‘treatment gap’ is much more significant in low- and middle-income countries. Without a formal diagnosis, patients do not receive the treatment and care they need, nor do they or their care-givers qualify for critical support programs. Early diagnosis and early intervention are two important means of narrowing the treatment gap. Thus, early diagnostic tools that can determine disease risk both quickly and accurately have significant therapeutic value on many levels. Research has confirmed that AD affects the brain long before actual symptoms of memory loss or cognitive decline actually manifest. To this date, however, there are no diagnostic tools for early detection; by the time a patient is diagnosed with AD using methods currently available, which involves subjective clinical assessment, often the pathological symptoms are already at an advanced state. As such, for the purpose of improving AD treatment and long term management, there exists an urgent need for developing new and effective methods for early diagnosis of AD or for detecting an increased risk of developing AD in a patient at a later time. This invention addresses this and other related needs by disclosing novel methods and kits related to the use of plasma or serum or whole blood protein markers or their combinations, to assess individual risk of developing Alzheimer's disease (AD).

### BRIEF SUMMARY OF THE INVENTION

The invention relates to the discovery of novel plasma protein markers associated with the Alzheimer's Disease (AD). The invention thus provides methods and compositions useful for diagnosis of AD as well as for indicating therapeutic efficacy of an agent for treating AD. As such, in a first aspect, the present invention provides a method for assessing a subject's risk of developing AD at a later time. The method includes the following steps: (1) comparing the subject's plasma or serum or whole blood level or concentration of any one protein selected from Tables 1-4 with a standard control level of the same protein found in the plasma or serum or whole blood, respectively, of an average healthy subject not suffering from or at increased risk for AD; (2) detecting that the subject's plasma or serum or whole blood level of the protein (which has a positive β value in Table 1, 2, 3, or 4) is higher than the standard control level, or that the subject' plasma or serum or whole blood level of the protein (which has a negative β value in Table 1, 2, 3, or 4) is lower than the standard control level; and (3) determining the subject as having increased risk for AD. While any of the 429 proteins identified in Table 2 is suitable for use in this method, in some cases the protein is selected from the 74 proteins set forth in Table 1, or from the 19 proteins set forth in Table 4, or from the 12 proteins set forth in Table 3. In some embodiments, the method also includes, prior to step (1), a step of measuring the plasma or serum or whole blood level of the protein. In some embodiments, the measuring step is proceeded by a step of obtaining a plasma or serum or whole blood sample from the subject. In some embodiments, when the subject is determined in step (3) as having increased risk for AD, the subject is then provided increased follow-up monitoring (e.g., monitoring tests at an increased frequency compared to the routine monitoring prescribed by a healthcare professional to a no-risk or low-risk person of similar age and medical background) or treatment as described in this disclosure.

In a second aspect, the present invention provides a method for assessing risk for Alzheimer's Disease (AD) among two subjects. The method includes these steps: (i) comparing the first subject's plasma or serum or whole blood level of any one protein selected from Tables 1-4 with the second subject's plasma or serum or whole blood level, respectively, of the same protein; (ii) detecting that the second subject's plasma or serum or whole blood level of the protein is higher than the first subject's plasma or serum or whole blood level, respectively, of the protein (which has a positive β value in Table 1, 2, 3, or 4), or that the second subject's plasma or serum or whole blood level of the protein is lower than the first subject's plasma or serum or whole blood level, respectively, of the protein (which has a negative β value in Table 1, 2, 3, or 4); and (iii) determining the second subject as having a higher risk to later develop AD than the first subject. While any of the 429 proteins identified in Table 2 is suitable for use in this method, in some embodiments the protein is selected from the 74 proteins set forth in Table 1, or from the 19 proteins set forth in Table 4, or from the 12 proteins set forth in Table 3. In some embodiments, the method further includes, a step of measuring the plasma or serum or whole blood level of the protein. In some embodiments, the measuring step is proceeded by a step of obtaining a plasma or serum or whole blood sample from the subject. In some embodiments, when a subject is determined in step (iii) as having a higher risk for AD, the subject is then given increased follow-up monitoring (e.g., monitoring tests at an increased frequency compared to the routine monitoring prescribed by a healthcare professional to a no-risk or low-risk person of similar age and medical background) or treatment as described in this disclosure, whereas the other subject, who is deemed to have a lower risk for AD, is subject to the routine monitoring prescribed by a healthcare professional to a no-risk or low-risk person of similar age and medical background.

In a third aspect, the present invention provides a kit for assessing risk for Alzheimer's Disease (AD) in a subject or for assessing therapeutic efficacy of a treatment regimen for AD. The kit includes at least one a reagent capable of determining the subject's plasma or serum or whole blood level or concentration of each one of any 5, 10, 15, or 20 proteins independently selected from the 429 proteins set forth in Table 2. In some embodiments, the proteins are independently selected from the 74 proteins set forth in Table 1, or the 19 proteins set forth in Table 4, or the 12 proteins set forth in Table 3. In some embodiments, the kit may in addition include a reagent capable of determining the subject's plasma or serum or whole blood level or concentration of each of amyloid β protein 42, amyloid β protein 40, and neurofilament light polypeptide (NfL). In some embodiments, the kit may further include a standard control for each of the proteins, reflecting the level/concentration of the same protein found in the plasma or serum or whole blood of an average healthy subject not suffering from or at increased risk for AD.

In a fourth aspect, the present invention provides a detection chip for assessing AD risk in a subject or for assessing therapeutic efficacy of a treatment regimen for AD. The chip comprises a solid substrate and a reagent capable of determining the subject's plasma or serum or whole blood level of each of any 5, 10, 15, or 20 proteins independently selected from the 429 proteins set forth in Table 2, with each reagent immobilized at an addressable location on the substrate. In some embodiments, the proteins are independently selected from the 74 proteins set forth in Table 1, or the 19 proteins set forth in Table 4, or the 12 proteins set forth in Table 3.

In a fifth aspect, the present invention provides a method for assessing risk for Alzheimer's Disease (AD) in a subject. The method includes these steps: (1) calculating a prediction score by inputting a set of values into the formula:

$$Individual AD prediction score = \frac{1}{1 + {e}^{- ( {β}_{i} Candidate {protein}_{i} + ε )}} ,$$

and (2) determining the subject who has a score from 0 to 0.25±0.05 as having low risk for AD, determining the subject who has a score from above 0.25±0.05 to 0.80±0.01 as having moderate risk for AD, and determining the subject who has a score from above 0.80±0.01 to 1 as having high risk for AD. In this method the set of values comprises the plasma or serum or whole blood level of each of the 12 proteins set forth in Table 3, and the weighted coefficients (βᵢ) and intercept (ε) of the proteins are set forth in Tables 5-8.

In some embodiments, the set of values consists of the plasma or serum or whole blood level of each of the 12 proteins in Table 3, the corresponding weighted coefficients (βᵢ) and intercept (ε) are set forth in Table 5, and the subject who has a score from 0 to 0.25 has low risk for AD; the subject who has a score from above 0.25 to 0.79 has moderate risk for AD; the subject who has a score from above 0.79 to 1 has high risk for AD.

In some embodiments, the set of values consists of the plasma or serum or whole blood level of each of the 19 proteins in Table 4, the corresponding weighted coefficients (βᵢ) and intercept (ε) are set forth in Table 6, and the subject who has a score from 0 to 0.21 has low risk for AD; the subject who has a score from above 0.21 to 0.8 has moderate risk for AD; the subject who has a score from above 0.8 to 1 has high risk for AD.

In some embodiments, the set of values consists of the ratio between plasma or serum or whole blood levels of amyloid β protein 42 and amyloid β protein 40, the plasma or serum or whole blood level of NfL, and the plasma or serum or whole blood level of each of the 12 proteins in Table 3, the corresponding weighted coefficients (βᵢ) and intercept (ε) are set forth in Table 7, and the subject who has a score from 0 to 0.20 has low risk for AD; the subject who has a score from above 0.20 to 0.80 has moderate risk for AD; the subject who has a score from above 0.80 to 1 has high risk for AD.

In some embodiments, the set of values consists of the ratio between plasma or serum or whole blood levels of amyloid β protein 42 and amyloid β protein 40, the plasma or serum or whole blood level of NfL, and the plasma or serum or whole blood level of each of the 19 proteins in Table 4, the corresponding weighted coefficients (βᵢ) and intercept (ε) are set forth in Table 8, and the subject who has a score from 0 to 0.30 has low risk for AD; the subject who has a score from above 0.30 to 0.80 has moderate risk for AD; the subject who has a score from above 0.80 to 1 has high risk for AD.

In some embodiments, the method further includes, prior to step (1), a step of measuring the plasma or serum or whole blood level of the proteins. In some embodiments, the method in additional includes, prior to the measuring step, another step of obtaining a plasma or serum or whole blood sample from the subject. In some embodiments, when the subject is determined in step (2) as having high risk for AD, the subject is then given increased follow-up monitoring (e.g., monitoring tests at an increased frequency compared to the routine monitoring prescribed by a healthcare professional to a no-risk or low-risk person of similar age and medical background) and treatment as described in this disclosure. When the subject is determined in step (2) as having moderate risk for AD, he is then given increased follow-up monitoring (e.g., monitoring tests at an increased frequency compared to the routine monitoring prescribed by a healthcare professional to a no-risk or low-risk person of similar age and medical background) as described in this disclosure. When the subject is determined as having low risk for AD, he is then given the routine monitoring generally prescribed by a physician to a no-risk or low-risk person for AD.

In a sixth aspect, the present invention provides a method for assessing relative risk for Alzheimer's Disease (AD) in two subjects. The method includes these steps: (i) calculating a prediction score for each of the two subjects by inputting a set of values into the formula:

$$Individual AD prediction score = \frac{1}{1 + {e}^{- ( {β}_{i} Candidate {protein}_{i} )}} ,$$

and (ii) determining the subject who has a higher score as having an higher risk for AD than the other subject. The set of values used in this method comprises the ratio between the plasma or serum or whole blood levels of amyloid β protein 42 and amyloid β protein 40, the plasma or serum or whole blood level of NfL, the plasma or serum or whole blood level of at least one of the proteins set forth in Table 2, and the corresponding weighted coefficients (βᵢ) are set forth in Table 1, 2, 3, 4, and 9.

In some embodiments, the set of values comprises the ratio between the plasma or serum or whole blood levels of amyloid β protein 42 and amyloid β protein 40, the plasma or serum or whole blood level of NfL, the plasma or serum or whole blood level of any combination of the proteins set forth in Table 2, and the corresponding weighted coefficients (βᵢ) are set forth in Table 1, 2, 3, 4, and 9.

In some embodiments, the set of values comprises the ratio between the plasma or serum or whole blood levels of amyloid β protein 42 and amyloid β protein 40, the plasma or serum or whole blood level of NfL, the plasma or serum or whole blood level of at least one of the proteins set forth in Table 1, 3, or 4, and the corresponding weighted coefficients (βᵢ) are set forth in Table 1, 3, 4, and 9.

In some embodiments, the set of values comprises the ratio between the plasma or serum or whole blood levels of amyloid β protein 42 and amyloid β protein 40, the plasma or serum or whole blood level of NfL, the plasma or serum or whole blood level of at least five of the proteins independently selected from Table 1, 3, or 4, and the corresponding weighted coefficients (βᵢ) are set forth in Table 1, 3, 4, and 9.

In some embodiments, the set of values comprises the ratio between the plasma or serum or whole blood levels of amyloid β protein 42 and amyloid β protein 40, the plasma or serum or whole blood level of NfL, the plasma or serum or whole blood level of at least ten of the proteins independently selected from Table 1, 3, or 4, and the corresponding weighted coefficients (βᵢ) are set forth in Table 1, 3, 4, and 9.

In some embodiments, the method further includes, prior to step (i), a step of measuring the plasma or serum or whole blood level of each of the proteins. In some embodiments, the method in addition includes, prior to the measuring step, a step of obtaining a plasma or serum or whole blood sample from the subjects. In some embodiments, when a subject is determined in step (ii) as having a higher risk for AD, the subject is then given increased follow-up monitoring (e.g., monitoring tests at an increased frequency compared to the routine monitoring prescribed by a healthcare professional to a no-risk or low-risk person of similar age and medical background) or treatment as described in this disclosure, whereas the other subject, who is deemed to have a lower risk for AD, is subject to the routine monitoring prescribed by a healthcare professional to a no-risk or low-risk person for AD.

In a seventh aspect, the present invention provides a method for assessing efficacy of a therapeutic agent for treating Alzheimer's Disease (AD) in a subject who has been diagnosed of AD. The method includes these steps: (1) comparing the subject's plasma or serum or whole blood levels of any one protein selected from Tables 1-4 before administration of the therapeutic agent with the subject's plasma or serum or whole blood levels of the protein after administration of the therapeutic agent; (2) detecting a decrease in the subject's plasma or serum or whole blood level of the protein (which has a positive β value in Table 1, 2, 3, or 4) or an increase in the subject' plasma or serum or whole blood level of the protein (which has a negative β value in Table 1, 2, 3, or 4) after administration of the therapeutic agent; and (3) determining the therapeutic agent as effective for treating AD. In some embodiments, the protein is selected from Table 1. In some embodiments, the protein is selected from Table 3. In some embodiments, the protein is selected from Table 4. In some embodiments, the method further includes, prior to step (1), a step of measuring the plasma or serum or whole blood level of the protein before and after administration. In some embodiments, the method may also include, prior to the measuring step, obtaining a plasma or serum or whole blood sample from the subject before and after administration.

In some embodiments, when the therapeutic agent is deemed in step (3) as effective for treating AD, the subject will continue his treatment by administration of the therapeutic agent; when the therapeutic agent is deemed in step (3) as not effective for treating AD, the subject will discontinue treatment by administration of the therapeutic agent; rather, the subject will initiate AD treatment by administration of a different therapeutic agent.

### BRIEF DESCRIPTION OF THE DRAWINGS

FIG. 1. Prediction of AD risk based on the model utilizing 12 plasma proteins. (a) Receiver operating characteristic (ROC) curve of the AD prediction model based on the plasma levels of 12 proteins (listed in Table 3) in the HK Chinese AD cohort. (b) Distribution of AD prediction scores stratified by phenotype (n=71 and 101 for NC and AD patients from the HK Chinese AD cohort, respectively). Predicted AD risk stages are defined by the distribution of AD prediction scores (Low: 0-0.25; Moderate: 0.25-0.79; High: 0.79-1.0).

FIG. 2. Prediction of AD risk based on the model utilizing 19 plasma proteins. (a) Receiver operating characteristic (ROC) curve of the AD prediction model based on the plasma levels of 19 proteins (listed in Table 4) in the HK Chinese AD cohort. (b) Distribution of AD prediction scores stratified by phenotype (n=71 and 101 for NC and AD patients from the HK Chinese AD cohort, respectively). Predicted AD risk stages are defined by the distribution of AD prediction scores (Low: 0-0.21; Moderate: 0.21-0.8; High: 0.8-1.0).

FIG. 3. Prediction of AD risk based on the model utilizing plasma Aβ_{42/40} ratio, plasma NfL and 12 plasma proteins. (a) Receiver operating characteristic (ROC) curve of the AD prediction model based on the plasma Aβ_{42/40} ratio, plasma NfL level and plasma levels of 12 proteins (listed in Table 3) in the HK Chinese AD cohort. (b) Distribution of AD prediction scores stratified by phenotype (n=71 and 101 for NC and AD patients from the HK Chinese AD cohort, respectively). Predicted AD risk stages are defined by the distribution of AD prediction scores (Low: 0-0.2; Moderate: 0.2-0.8; High: 0.8-1.0).

FIG. 4. Prediction of AD risk based on the model utilizing plasma Aβ_{42/40} ratio, plasma NfL and 19 plasma proteins. (a) Receiver operating characteristic (ROC) curve of the AD prediction model based on the plasma Aβ_{42/40} ratio, plasma NfL level and plasma levels of 19 proteins (listed in Table 4) in the HK Chinese AD cohort. (b) Distribution of AD prediction scores stratified by phenotype (n=71 and 101 for NC and AD patients from the HK Chinese AD cohort, respectively). Predicted AD risk stages are defined by the distribution of AD prediction scores (Low: 0-0.3; Moderate: 0.3-0.8; High: 0.8-1.0).

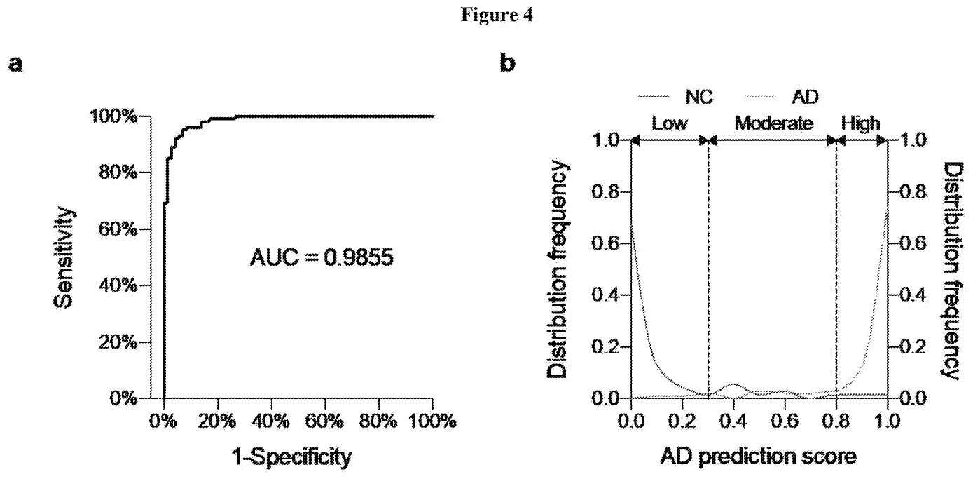

### DEFINITIONS

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

In this disclosure the term “biological sample” or “sample” includes sections of tissues such as biopsy and autopsy samples, and frozen sections taken for histologic purposes, or processed forms of any of such samples. Biological samples include blood and blood fractions or products (e.g., whole blood, acellular fraction of blood (serum, plasma), and blood cells), sputum or saliva, lymph and tongue tissue, cultured cells, e.g., primary cultures, explants, and transformed cells, stool, urine, stomach biopsy tissue etc. A biological sample is typically obtained from a eukaryotic organism, which may be a mammal, may be a primate and may be a human subject.

The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains.

The term “antibody” also refers to antigen- and epitope-binding fragments of antibodies, e.g., Fab fragments, that can be used in immunological affinity assays. There are a number of well characterized antibody fragments. Thus, for example, pepsin digests an antibody C-terminal to the disulfide linkages in the hinge region to produce F(ab)′2, a dimer of Fab which itself is a light chain joined to V_{H}-C_{H}1 by a disulfide bond. The F(ab)′2 can be reduced under mild conditions to break the disulfide linkage in the hinge region thereby converting the (Fab′)₂ dimer into an Fab′ monomer. The Fab′ monomer is essentially a Fab with part of the hinge region (see, e.g., Fundamental Immunology, Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody also includes antibody fragments either produced by the modification of whole antibodies or synthesized using recombinant DNA methodologies.

The phrase “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background. On the other hand, the term “specifically bind” when used in the context of referring to a polynucleotide sequence forming a double-stranded complex with another polynucleotide sequence describes “polynucleotide hybridization” based on the Watson-Crick base-pairing, as provided in the definition for the term “polynucleotide hybridization method.”

As used in this application, an “increase” or a “decrease” refers to a detectable positive or negative change in quantity from a comparison control, e.g., an established standard control (such as an average level/amount of a particular protein found in samples from healthy subjects who has not been diagnosed with AD and has no increased risk for AD). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the protein or used to detect antibodies specifically reactive with the protein. Typically a detectable label is attached to a probe or a molecule with defined binding characteristics (e.g., an antibody with a known binding specificity to a polypeptide antigen), so as to allow the presence of the probe (and therefore its binding target) to be readily detectable.

The term “amount” as used in this application refers to the quantity of a substance of interest, such as a polypeptide of interest, present in a sample. Such quantity may be expressed in the absolute terms, i.e., the total quantity of the substance in the sample, or in the relative terms, i.e., the concentration of the substance in the sample.

The term “subject” or “subject in need of treatment,” as used herein, includes individuals who seek medical attention due to risk of (e.g., with family history), or having been diagnosed of, AD. Subjects also include individuals currently undergoing therapy that seek manipulation of the therapeutic regimen. Subjects or individuals in need of treatment include those that demonstrate symptoms of AD or are at risk of suffering from AD or its symptoms. For example, a subject in need of treatment includes individuals with a genetic predisposition or family history for AD, those that have suffered relevant symptoms in the past, those that have been exposed to a triggering substance or event, as well as those suffering from chronic or acute symptoms of the condition. A “subject in need of treatment” may be at any age of life.

“Inhibitors,” “activators,” and “modulators” of a target protein are used to refer to inhibitory, activating, or modulating molecules, respectively, identified using in vitro and in vivo assays for the protein binding or signaling, e.g., ligands, agonists, antagonists, and their homologs and mimetics. The term “modulator” includes inhibitors and activators. Inhibitors are agents that, e.g., partially or totally block, decrease, prevent, delay activation, inactivate, desensitize, or down regulate the activity of the target protein. In some cases, the inhibitor directly or indirectly binds to the protein, such as a neutralizing antibody. Inhibitors, as used herein, are synonymous with inactivators and antagonists. Activators are agents that, e.g., stimulate, increase, facilitate, enhance activation, sensitize or up regulate the activity of the target protein. Modulators include the target protein's ligands or binding partners, including modifications of naturally-occurring ligands and synthetically-designed ligands, antibodies and antibody fragments, antagonists, agonists, small molecules including carbohydrate-containing molecules, siRNAs, RNA aptamers, and the like.

The term “treat” or “treating,” as used in this application, describes an act that leads to the elimination, reduction, alleviation, reversal, prevention and/or delay of onset or recurrence of any symptom of a predetermined medical condition. In other words, “treating” a condition encompasses both therapeutic and prophylactic intervention against the condition.

The term “effective amount,” as used herein, refers to an amount that produces therapeutic effects for which a substance is administered. The effects include the prevention, correction, or inhibition of progression of the symptoms of a disease/condition and related complications to any detectable extent. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “standard control,” as used herein, refers to a sample comprising an analyte of a predetermined amount to indicate the quantity or concentration of this analyte present in this type of sample (e.g., a predetermined DNA/mRNA or protein) taken from an average healthy subject not suffering from or at risk of developing a predetermined disease or condition (e.g., Alzheimer's Disease). When used in the context of describing a value, this term may also be used to simply refer to the quantity or concentration of this analyte present in a “standard control” sample.

The term “average,” as used in the context of describing a healthy subject who does not suffer from and is not at risk of developing a relevant disease or disorders (e.g., AD) refers to certain characteristics, such as the level of a pertinent protein in the person's sample (e.g., serum or plasma or whole blood), that are representative of a randomly selected group of healthy humans who are not suffering from and is not at risk of developing the disease or disorder. This selected group should comprise a sufficient number of human subjects such that the average amount or concentration of the analyte of interest among these individuals reflects, with reasonable accuracy, the corresponding profile in the general population of healthy people. Optionally, the selected group of subjects may be chosen to have a similar background to that of a person whose is tested for indication or risk of the relevant disease or disorder, for example, matching or comparable age, gender, ethnicity, and medical history, etc.

The term “inhibiting” or “inhibition,” as used herein, refers to any detectable negative effect on a target biological process or on the level of a biomarker (e.g., a protein). Typically, an inhibition is reflected in a decrease of at least 10%, 20%, 30%, 40%, or 50% in one or more parameters indicative of the biological process or its downstream effect or the level of biomarker when compared to a control where no such inhibition is present. The term “enhancing” or “enhancement” is defined in a similar manner, except for indicating a positive effect, i.e., the positive change is at least 10%, 20%, 30%, 40%, 50%, 80%, 100%, 200%, 300% or even more in comparison with a control. The terms “inhibitor” and “enhancer” are used to describe an agent that exhibits inhibiting or enhancing effects as described above, respectively. Also used in a similar fashion in this disclosure are the terms “increase,” “decrease,” “more,” and “less,” which are meant to indicate positive changes in one or more predetermined parameters by at least 10%, 20%, 30%, 40%, 50%, 80%, 100%, 200%, 300% or even more, or negative changes of at least 10%, 20%, 30%, 40%, 50%, 80% or even more in one or more predetermined parameters.

As used herein, the term “Chinese” refers to ethnic Chinese people who and whose ancestors have been residing in the historical territories of China, including the mainland and Hong Kong, for a length of time, e.g., at least the last 3, 4, 5, 6, 7, or 8 generations or the last 100, 150, 200, 250, or 300 years.

### DETAILED DESCRIPTION OF THE INVENTION

### I. Introduction

Alzheimer' disease (AD) is one of the most common forms of dementia in the world, accounting for 60-70% of all dementia cases. It is an irreversible degenerative brain disease and a leading cause of mortality among the elderly. The hallmarks of this disease are deposition of extracellular β-amyloid (Aβ) plaques and intracellular neurofibrillary tangles, which result in declining memory, reasoning, judgment, and locomotion abilities, with symptoms worsening over time.

Currently, an estimated 35 million people worldwide are afflicted with AD. This figure is expected to rise significantly to 100 million by 2050 due to longer life expectancies. There is no cure for AD; and the pathophysiology of the disease is still relatively unknown. There are only five drugs approved by the US Food and Drug Administration (FDA) to treat AD, but these only alleviate symptoms rather than alter disease pathology, as they cannot reverse the condition or prevent further deterioration, and are ineffective in severe conditions. Thus, early diagnosis and early therapeutic intervention is critical in the management of AD. Research has confirmed that AD affects the brain long before actual symptoms of memory loss or cognitive decline actually manifest. To this date, however, there are no effective and reliable diagnostic tools for early detection of AD; by the time a patient is diagnosed with AD using standard methods currently in use, which involves subjective clinical assessment, the pathological symptoms are already at an advanced stage. The present disclosure provides high performance diagnostic methods utilizing one or more protein markers for assessing AD risk to aid early diagnosis.

### II. Quantitation of Marker Proteins

### A. Obtaining Samples

The first step of practicing the present invention is to obtain a blood sample from a subject being tested for assessing the risk of developing AD or monitoring for AD severity or progression. Samples of the same type should be taken from both a control group (normal individuals not suffering from AD and without increased risk for AD) and a test group (subjects being tested for possible AD or for increased risk for AD, for example). Standard procedures routinely employed in hospitals or clinics are typically followed for this purpose.

For the purpose of detecting the presence/quantity of marker proteins or assessing the risk of developing AD in test subjects, individual patients' blood samples are taken, and the serum/plasma or whole blood level of pertinent marker proteins (e.g., amyloid β protein 40, amyloid β protein 42, NfL, or one or more proteins identified in Tables 1-4) may be measured and then compared to a standard control. If an increase or a decrease in the level of one or more of these marker proteins (depending on the protein's β value provided in Tables 1-4) is observed when compared to the control level, the test subject is deemed to have AD or have an elevated risk of developing later developing the condition. For the purpose of monitoring disease progression or assessing therapeutic effectiveness in AD patients, individual patient's blood samples may be taken at different time points, such that the level of individual marker protein(s) can be measured to provide information indicating the state of disease. For instance, when a patient's maker protein level shows a general trend of increasing or decreasing over time, the patient is deemed to be improving in the severity of AD or the therapy the patient has been receiving is deemed effective (depending on the specific β value of the protein maker as shown in the Tables). A lack of substantial change in a patient's marker protein level would indicate a lack of change in the status of AD and ineffectiveness of the therapy given to the patient.

Moreover, the present inventors have devised novel calculation methods to produce a composite risk score based on multiple marker protein levels (e.g., amyloid β protein 40, amyloid β protein 42, NfL, or one or more proteins identified in Tables 1-4) to assess the AD risk of an individual or to assess the relative AD risk between two or more individuals.

### B. Preparing Samples for Protein Detection

The blood sample from a subject is suitable for the present invention and can be obtained by well-known methods and as described in standard medical literature. In certain applications of this invention, serum or plasma or whole blood may be the preferred sample type. In other cases, whole blood samples may be used.

A blood sample is obtained from a person to be tested or monitored for AD using a method of the present invention. Collection of blood sample from an individual is performed in accordance with the standard protocol hospitals or clinics generally follow. An appropriate amount of blood is collected and may be stored according to standard procedures prior to further preparation.

The analysis of marker protein(s) found in a patient's sample according to the present invention may be performed using, e.g., serum or plasma or whole blood. The methods for preparing patient samples for protein extraction/quantitative detection are well known among those of skill in the art.

### C. Determining the Level of Marker Proteins

A protein of any particular identity, such as amyloid β protein 40, amyloid β protein 42, NfL, or any one identified in Tables 1-4, can be detected using a variety of immunological assays. In some embodiments, a sandwich assay can be performed by capturing the protein from a test sample with an antibody having specific binding affinity for the protein. The protein then can be detected with a labeled antibody having specific binding affinity for it. Such immunological assays can be carried out using microfluidic devices such as microarray protein chips. A protein of interest (e.g., amyloid β protein 40, amyloid β protein 42, NfL, or one or more proteins identified in Tables 1-4) can also be detected by gel electrophoresis (such as 2-dimensional gel electrophoresis) and western blot analysis using specific antibodies. Alternatively, standard immunohistochemical techniques can be used to detect a given protein (e.g., amyloid β protein 40, amyloid β protein 42, NfL, or one or more proteins identified in Tables 1-4), using the appropriate antibodies. Both monoclonal and polyclonal antibodies (including antibody fragment with desired binding specificity) can be used for specific detection of the polypeptide. Such antibodies and their binding fragments with specific binding affinity to a particular protein (e.g., amyloid β protein 40, amyloid β protein 42, NfL, or one or more proteins identified in Tables 1-4) can be generated by known techniques.

Other methods may also be employed for measuring the level of marker protein(s) in practicing the present invention. For instance, a variety of methods have been developed based on the mass spectrometry technology to rapidly and accurately quantify target proteins even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009 February; 8(2):787-797.

### III. Establishing a Standard Control

In order to establish a standard control for practicing the method of this invention, a group of healthy persons free of AD or increased risk for developing AD as conventionally defined is first selected. These individuals are within the appropriate parameters, if applicable, for the purpose of screening for and/or monitoring AD using the methods of the present invention. Optionally, the individuals are of same gender, similar age, or similar ethnic background to the test subjects.

The healthy status of the selected individuals is confirmed by well-established, routinely employed methods including but not limited to general physical examination of the individuals and general review of their medical history.

Furthermore, the selected group of healthy individuals must be of a reasonable size, such that the average amount/concentration of marker protein(s) in the serum or plasma or whole blood sample obtained from the group can be reasonably regarded as representative of the normal or average level among the general population of healthy people without AD or increased risk for AD. Preferably, the selected group comprises at least 10, 20, 30, or 50 human subjects.

Once an average value for the marker protein(s) is established based on the individual values found in each subject of the selected healthy control group, this average or median or representative value or profile is considered a standard control. A standard deviation is also determined during the same process. In some cases, separate standard controls may be established for separately defined groups having distinct characteristics such as age, gender, or ethnic background.

### IV. Monitoring and Treatment

In a related aspect, the present invention also provides treatment methods for AD patients upon detection of AD or a heightened risk of later developing AD in a patient. In some embodiments, the method comprises, upon determining a subject as having an increased risk for AD, administering a treatment to said subject, for example, an acetylcholinesterase inhibitor (such as donepezil, galantamine, rivastigmine), memantine, a glutamate receptor blocker, citalopram, fluoxetine, paroxeine, sertraline, trazodone, lorazepam, oxazepam, aripiprazole, clozapine, haloperidol, olanzapine, quetiapine, risperidone, ziprasidone, nortriptyline, tricyclic antidepressants, benzodiazepines, temazepam, zolpidem, zaleplon, chloral hydrate, coenzyme Q10, ubiquinone, coral calcium, Ginkgo biloba, huperzine A, omega-3 fatty acids, phosphatidylserine, or any combination thereof.

In some cases, when the diagnostic method steps described above and herein are completed, optionally with additional diagnostic examination performed to provide further confirmatory information (for example, by brain imaging via CT scan or other imaging techniques to show excessive loss of brain volume, or by testing cognitive capability to show an accelerated decline), and a patient has been determined to either already have AD or is at a significantly increased risk of later developing AD, suitable therapeutic or prophylactic regimens may be ordered by physicians or other medical professionals to treat the patient, to manage/alleviate the ongoing symptoms, or to delay the future onset of the disease. The U.S. Food and Drug Administration (FDA) has approved a number of cholinesterase inhibitors, including donepezil (Aricept™, the only cholinesterase inhibitor approved to treat all stages of AD, including moderate to severe), rivastigmine (Exelon™, approved to treat mild to moderate AD), galantamine (Razadyne™, mild to moderate patients) and memantine (Namenda™). Donepezil is the only cholinesterase inhibitor approved to treat all stages of AD, including moderate to severe. Any one or more of these drugs can be prescribed for treating patients who have been diagnosed with AD in accordance with the methods of this invention. Another possibility of treatment is administration of trazodone, which is currently approved for use as an antidepressant and has been reported as an effective agent for ameliorating AD symptoms.

For patients who are deemed at high or increased risk for developing AD in a future time but do not yet exhibit any clinical symptoms, continuous monitoring is also appropriate, especially at an increased frequency. For example, the patients may be subject to more frequently scheduled regular testing (e.g., once every six months, once a year, or once every two years) to detect any accelerated change in their cognitive capabilities. Methods suitable for such regular monitoring include General Practitioner Assessment of Cognition (GPCOG), Mini-Cog, Eight-item Informant Interview to Differentiate Aging and Dementia (AD8), and Short Informant Questionnaire on Cognitive Decline in the Elderly (IQCODE). Furthermore, prophylactic treatment with trazodone may also be recommended.

### V. Kits and Devices

The invention provides compositions and kits for practicing the methods described herein to assess the pertinent marker protein level in a subject's serum/plasma or whole blood, which can be used for various purposes such as detecting or diagnosing the presence of AD, determining the risk of developing the condition, and monitoring progression of the condition in a patient, including assessing the therapeutic efficacy of a therapy administered for the condition among patients who have received a diagnosis of the disease and have undergone treatment.

Kits for carrying out assays for determining marker protein levels typically include at least one antibody useful for specific binding to the marker protein amino acid sequence. Optionally, this antibody is labeled with a detectable moiety. The antibody can be either a monoclonal antibody or a polyclonal antibody. In some cases, the kits may include at least two different antibodies, one for specific binding to a marker protein (i.e., the primary antibody) and the other for detection of the primary antibody (i.e., the secondary antibody), which is often attached to a detectable moiety.

Typically, the kits also include an appropriate standard control. The standard controls indicate the average value of marker protein(s) in the serum or plasma or whole blood of healthy subjects not suffering from or at increased risk of developing AD. In some cases, such standard control may be provided in the form of a set value. In addition, the kits of this invention may provide instruction manuals to guide users in analyzing test samples and assessing the presence or risk of AD, or disease status/progression in a test subject.

In a further aspect, the present invention can also be embodied in a device or a system comprising one or more such devices, which is capable of carrying out all or some of the method steps described herein. For instance, in some cases, the device or system performs the following steps upon receiving a serum or plasma or whole blood sample taken from a subject being tested for detecting AD, assessing the risk of developing AD, or assessing the disease status/progression: (a) determining in sample the amount or concentration of marker protein; (b) comparing the amount/concentration with a standard control value; and (c) providing an output indicating whether AD is present in the subject or whether the subject is at increased risk of developing AD, or whether the patient has a higher risk of later developing AD relative to another patient being tested. In other cases, the device or system of the invention performs the task of steps (b) and (c), after step (a) has been performed and the amount or concentration from (a) has been entered into the device. Preferably, the device or system is partially or fully automated.

### EXAMPLES

The following examples are provided by way of illustration only and not by way of limitation. Those of skill in the art will readily recognize a variety of non-critical parameters that could be changed or modified to yield essentially the same or similar results.

### Introduction

Alzheimer's disease (AD) is the most common neurodegenerative diseases that mainly affects individuals over the age of 65. It is characterized by the accumulation of amyloid beta (Aβ) plaques and neurofibrillary tangles of tau protein, together with synaptic dysfunction and neuronal loss in the brain². Disease symptoms include memory loss, impaired reasoning and judgement, and reduced locomotion abilities³. There are an estimated 47 million people worldwide afflicted with the disease and this figure is expected to rise to 132 million by 2050⁴. However, due to the incomplete understanding and delayed diagnosis of the disease, there is no cure yet, making AD one of the top threats to public health worldwide.

Currently, AD diagnosis is mostly limited to reviewing medical history, standardized memory tests, and physician expertise, which is arguably subjective. The adoption of imaging techniques such as magnetic resonance imaging (MRI) and positron-emission tomography (PET), which detects the structural changes and the presence of the AD-associated biomarkers Aβ and tau in the brains, and proteomic techniques for measuring cerebrospinal fluid (CSF) levels of Aβ, tau, and neurofilament light polypeptide (NfL) is enabling more accurate diagnosis and classification of the disease⁵. However, the high costs of MRI and PET as well as the invasive nature of lumbar punctures for CSF collection preclude them from routine clinical examination, and thus impedes their use for early diagnosis of AD. With the increasing number of AD cases around the world, it is critical to develop less invasive and more cost-effective diagnostic techniques to facilitate efficient AD screening and classification of patients at population-scale.

A blood-based test for AD would be an ideal solution under this circumstance. Recent investigations have shown that the altered AD-associated biomarker levels (Aβ_{42/40} ratio, tau, and NfL) in the blood of AD patients are indicative of disease pathology, and may be leveraged for diagnostic purposes⁶. Nevertheless, none of these biomarkers have sufficient diagnostic precision, which limits their potential for clinical use⁷. One of the essential reasons is that the peripheral blood system is more complicated in composition and is affected by not only the brain but also other body systems such as the peripheral, immune, cardiovascular, and metabolic systems. Thus, the existing AD-associated biomarkers are unable to adequately capture the disease-associated phenotypic changes in blood. Indeed, studies have shown that cytokines and angiogenic proteins also have altered plasma levels in AD, and several of them have been experimentally validated for their contribution to AD pathology⁸. Therefore, developing an accurate and sensitive blood-based diagnostic test for AD requires a more comprehensive proteomic study to fully capture the AD plasma signatures.

In this study, in addition to measuring the plasma levels of AD-associated biomarkers (Aβ and NfL), the present inventors further measured the levels of 429 plasma proteins in samples collected from 180 elderly people from a Hong Kong Chinese AD cohort. By integrating the plasma levels of these AD-associated proteins, the inventors have developed AD prediction models that, to a great extent, differentiate AD patients from normal controls (NC). These findings collectively provide a high-performance blood-based strategy for assessing AD risks.

### Materials and Methods

Subject Recruitment for the Hong Kong Chinese AD cohort: A cohort of Hong Kong Chinese participants who visited the Specialist Outpatient Department of the Prince of Wales Hospital, the Chinese University of Hong Kong, were recruited (n=106 and 74 for AD and normal controls [NC], respectively). All participants were ≥60 years old. The clinical diagnosis of AD was established on the basis of the American Psychiatric Association's Diagnostic and Statistical Manual of Mental Disorders, Fifth Edition (DSM-5)⁹.

All participants were subjected to medical history assessment, the Montreal Cognitive Assessment (MoCA) for cognitive and functional assessment, and neuroimaging assessment by MRI¹⁰. Each individual's data including age, sex, education, medical history, cardiovascular disease history, brain region volume, and white blood cell counts were recorded. Individuals with any significant neurologic disease or psychiatric disorder were excluded. This study was approved by the Prince of Wales Hospital of the Chinese University of Hong Kong as well as the Hong Kong University of Science and Technology. All participants provided written informed consent for both study participation and sample collection.

DNA and plasma extraction from blood samples: K3EDTA tubes (VACUETTE) were used to collect the whole blood (3 mL) from participants. Blood samples were centrifuged at 2,000×g for 15 min to separate the cell pellet and plasma. The plasma was collected, aliquoted, and stored at −80° C. until use. The cell pellets were sent to the Centre for PanorOmic Science (Genomics and Bioinformatics Cores, University of Hong Kong, Hong Kong, China) for genomic DNA extraction using the QIAsymphony DSP DNA Midi Kit (QIAGEN) on a QIAsymphony SP platform (QIAGEN). Genomic DNA was eluted with water or Elution Buffer ATE (QIAGEN) and stored at 4° C. DNA concentration was determined by BioDrop μLITE+ (BioDrop).

Detection of plasma proteins: The plasma levels of 429 proteins were measured by Olink biomarker panels including Cardiometabolic, Cardiovascular II, Cardiovascular III, Cell regulation, Development, Immune response, Inflammation, Metabolism, Neuro exploratory, Neurology, Oncology II, Oncology III, and Organ damage. The plasma levels of the “ATN” biomarkers (i.e., Aβ_{40/42}, tau, and neurofilament light polypeptide [NfL]) were measured by the Quanterix NF-light Simoa Assay Advantage Kit and the Neurology 3-Plex A Kit.

Whole-genome sequencing, variant calling and principal component analysis: DNA samples of participants were submitted to Novogene for library construction and WGS. Samples were sequenced on an Illumina Hiseq X (average depth: 5×). Genomic regions covering 500 kilobases up- and downstream of candidate variants were analyzed using the GotCloud pipeline¹¹. Genotype results stored in VCF files were used for principal component analysis. The top five principal components were generated by PLINK software with the following parameters: —pca header tabs, —maf 0.05, —hwe 0.00001, and —not-chr x y.

Analysis of the association between plasma proteins and AD: The R rntransform function from the GenABEL package was used to normalize plasma protein levels based on rank. The alteration of the plasma proteins in AD was determined on the basis of the association between normalized protein levels and AD phenotype, adjusting for age, sex, disease history, and population structure (i.e., the top five principal components) using the following linear model (βᵢ, the weighted coefficient for corresponding factors; E, the intercept of the linear equation):

Normalized protein level˜β₁AD+β₂Age+,β₃Sex+βᵢDiseaseᵢ+βⱼPCⱼ+ε

Generation of AD prediction scores: For each prediction model, the weighted coefficient (βᵢ) of corresponding candidate proteins and intercept (ε) were generated by fitting the plasma levels of candidate proteins and AD phenotype information of participants in the discovery cohort into logistic regression model using the following formula:

$$Phenotype ( AD = 1 , NC = 0 ) = \frac{1}{1 + {e}^{- ( {β}_{i} Candidate {protein}_{i} + ε )}}$$

Individual AD prediction scores were calculated on the basis of the plasma levels of candidate proteins and corresponding weighted coefficient (βᵢ) and intercept (ε) using the following linear model:

$$Individual AD prediction score = \frac{1}{1 + {e}^{- ( {β}_{i} Candidate {protein}_{i} + ε )}}$$

The predicted AD risk stages were defined by the distribution of AD prediction scores, separated into low risk, moderate risk and high risk groups.

Evaluation of prediction accuracy: The R plot.roc and auc functions were used to generate the receiver operating characteristic (ROC) curves and corresponding areas under the curve (AUCs) of prediction models for AD risk prediction. The prediction accuracy of models was denoted by the value of AUCs.

Statistical analysis and data visualization. The investigators who performed the protein detection were blinded to the phenotypes of the human participants. The significance of the associations among candidate factors in human participants was assessed by linear regression analysis, adjusting for age, sex, disease history, and population structure (i.e., the top five principal components obtained from the principal component analysis using whole-genome sequencing data). The level of significance was set at P<0.05. All other statistical plots were generated using GraphPad Prism version 8.0.

### Example I: Models Using Individual Plasma Protein in Assessing AD Risks

The levels of 429 plasma proteins (Table 2) in samples collected from the HK Chinese AD cohort (n=180) were measured. These 429 plasma proteins all displayed significant changes in AD in comparison to NC (p<0.05; Table 2). In particular, 74 novel plasma proteins displayed strong alteration in AD (Table 1). Based on the altered plasma levels of the 74 or 429 plasma proteins in AD patients, an assessing tool was developed for comparing AD risks between individuals using information from plasma proteins. An individual will have higher AD risks, if the individual has higher plasma level of the proteins that elevated in AD blood (β>0) or lower plasma level of the proteins that reduced in AD blood (β<0; Table 1, 2)

### Example II: Model by Integrating 12 or 19 Plasma Proteins in Predicting AD Risks

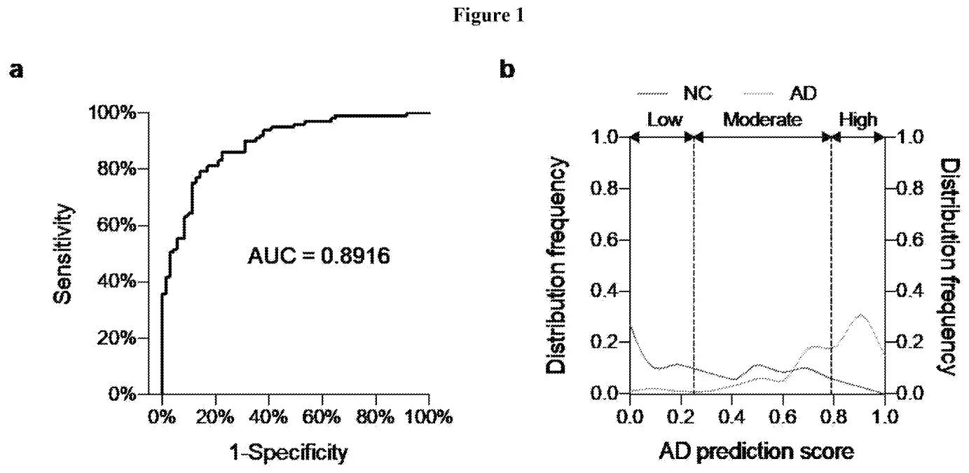

By integrating the plasma levels of the 12 proteins (i.e., CD164, CETN2, GAMT, GSAP, hK14, LGMN, NELL1, PRDX1, PRKCQ, TMSB10, VAMPS and VPS37A; Table 3), the present inventors developed a mixed prediction model that accurately predicted AD risks (AUC=0.8916; FIG. 1a). An AD risk scoring system was established by assigning individuals with AD prediction scores. The resulting scores distinguished the NC and AD patients (Table 5 and FIG. 1b). Based on the predicted scores, three AD risk stages were further proposed to predict disease risks. Individuals with AD prediction scores lower than 0.25 will have low AD risks. By comparison, individuals with the scores in range of 0.25 to 0.79 or with the scores larger than 0.79 will have moderate or high risks for AD, respectively.

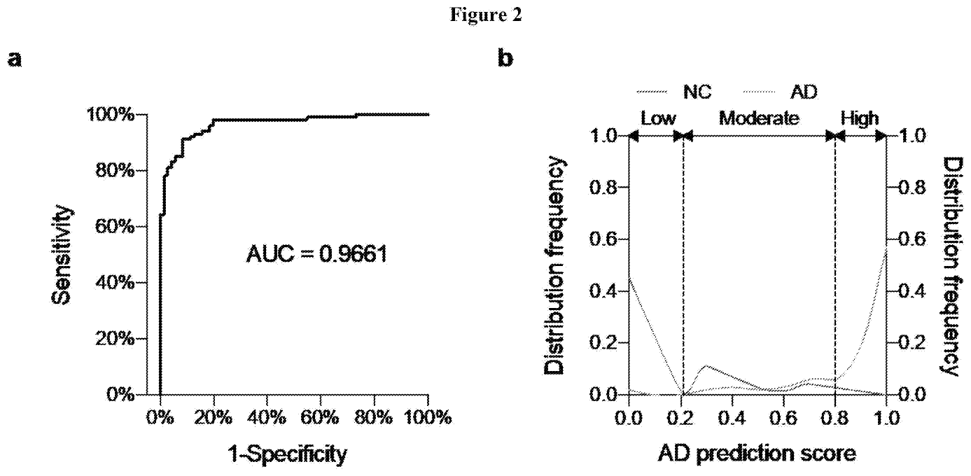

By further integrating the plasma levels of the 7 plasma proteins (i.e., AOC3, CASP-3, CD8A, KLK4, LIF-R, LYN, and NFKBIE) into the 12-protein model (Table 4), the inventors developed a mixed prediction model that further improved the prediction for AD risks (AUC=0.9661; FIG. 2a). The AD prediction scores better distinguished the NC and AD patients (Table 6 and FIG. 2b). Individuals with AD prediction scores lower than 0.21 will have low AD risks. By comparison, individuals with the scores in range of 0.21 to 0.8 or with the scores larger than 0.8 will have moderate or high risks for AD, respectively.

### Example III: Combined Model of Plasma AN Biomarkers and 12 or 19 Plasma Proteins in Predicting AD Risks

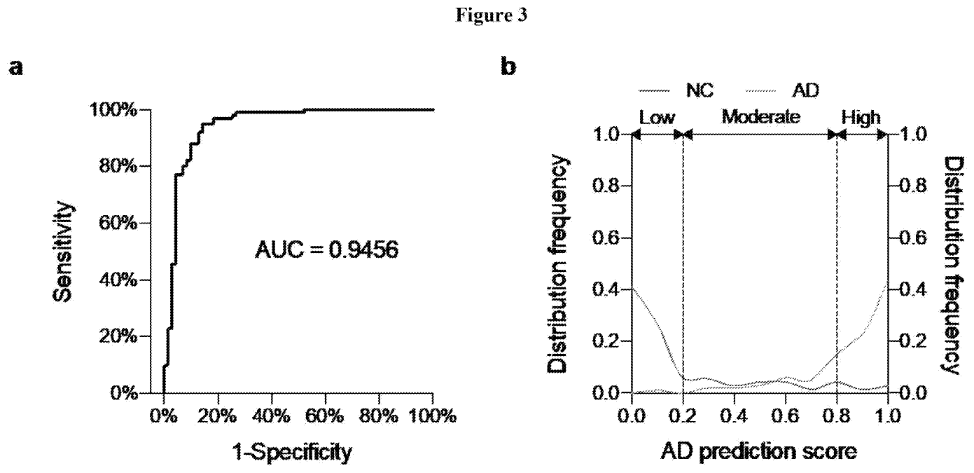

The combined prediction models were then developed by integrating the plasma Aβ_{42/40} ratio and plasma NfL level (AN) into the 12-protein or 19-protein model. Both combined models improved the AD prediction (AUC=0.9456 and 0.9855 for AN+12 proteins and AN+19 proteins, respectively; FIG. 3a, 4a). Moreover, the two combined models generated AD prediction scores that clearly separated NC and AD patients (Table 7-8 and FIG. 3b, 4b). For the model utilizing AN and 12 proteins, individuals with AD prediction scores lower than 0.2, in the range of 0.2-0.8 and larger than 0.8 will have low, moderate and high AD risks, respectively. For the model utilizing AN and 19 proteins, individuals with AD prediction scores lower than 0.3, in the range of 0.3-0.8 and larger than 0.8 will have low, moderate and high AD risks, respectively. Collectively, these results showed that the AD risk prediction models we developed takes full advantages of the effects of each candidate plasma protein in disease pathology, and can serve as a high-performance strategy for prediction of AD risks.

All patents, patent applications, and other publications, including GenBank Accession Numbers and equivalents, cited in this application are incorporated by reference in the entirety for all purposes.

TABLE 1
List of 74 plasma proteins associated with AD phenotypes. β, effect size.

| Protein name | Uniprot ID | β | Fold Change | P-value |
|---|---|---|---|---|
| EIF4G1 | Q04637 | −1.396 | 0.257 | 5.44E−21 |
| PLXNA4 | Q9HCM2 | −1.476 | 0.286 | 1.10E−20 |
| SNAP29 | O95721 | −1.397 | 0.357 | 3.61E−20 |
| BCR | P11274 | −1.468 | 0.329 | 7.57E−20 |
| PPP1R9B | Q96SB3 | −1.426 | 0.280 | 7.61E−20 |
| TXLNA | P40222 | −1.491 | 0.353 | 9.90E−20 |
| BANK1 | Q8NDB2 | −1.416 | 0.189 | 1.01E−19 |
| ARHGEF12 | Q9NZN5 | −1.420 | 0.244 | 1.70E−19 |
| INPPL1 | O15357 | −1.458 | 0.209 | 3.83E−19 |
| CLIP2 | Q9UDT6 | −1.470 | 0.198 | 7.51E−19 |
| TDRKH | Q9Y2W6 | −1.424 | 0.322 | 1.01E−18 |
| NEMO | Q9Y6K9 | −1.390 | 0.325 | 1.30E−18 |
| MESDC2 | Q14696 | −1.453 | 0.376 | 1.51E−18 |
| STK4 | Q13043 | −1.395 | 0.216 | 1.65E−18 |
| ITGB1BP2 | Q9UKP3 | −1.469 | 0.300 | 1.65E−18 |
| CALCOCO1 | Q9P1Z2 | −1.369 | 0.216 | 1.94E−18 |
| SRPK2 | P78362 | −1.426 | 0.484 | 2.11E−18 |
| DAPP1 | Q9UN19 | −1.405 | 0.174 | 2.14E−18 |
| DAB2 | P98082 | −1.368 | 0.389 | 2.23E−18 |
| ZBTB16 | Q05516 | −1.442 | 0.475 | 2.90E−18 |
| SRC | P12931 | −1.458 | 0.208 | 4.82E−18 |
| SNAP23 | O00161 | −1.369 | 0.224 | 4.85E−18 |
| MAP4K5 | Q9Y4K4 | −1.463 | 0.181 | 5.14E−18 |
| ERBB2IP | Q96RT1 | −1.394 | 0.304 | 8.00E−18 |
| YES1 | P07947 | −1.436 | 0.237 | 8.69E−18 |
| SH2B3 | Q9UQQ2 | −1.422 | 0.273 | 1.04E−17 |
| FKBP1B | P68106 | −1.381 | 0.398 | 1.11E−17 |
| WASF1 | Q92558 | −1.442 | 0.320 | 1.17E−17 |
| AIFM1 | O95831 | −1.330 | 0.371 | 1.21E−17 |
| MAP2K6 | P52564 | −1.373 | 0.448 | 1.23E−17 |
| PRTFDC1 | Q9NRG1 | −1.393 | 0.246 | 1.39E−17 |
| CDKN1A | P38936 | −1.410 | 0.287 | 1.56E−17 |
| PMVK | Q15126 | −1.443 | 0.203 | 1.70E−17 |
| FOXO1 | Q12778 | −1.453 | 0.385 | 2.52E−17 |
| USO1 | O60763 | −1.418 | 0.270 | 3.11E−17 |
| HEXIM1 | O94992 | −1.331 | 0.428 | 5.64E−17 |
| GOPC | Q9HD26 | −1.480 | 0.284 | 5.65E−17 |
| TBCB | Q99426 | −1.374 | 0.236 | 8.61E−17 |
| TACC3 | Q9Y6A5 | −1.362 | 0.416 | 4.38E−16 |
| NFATC1 | O95644 | −1.383 | 0.435 | 4.90E−16 |
| LAT2 | Q9GZY6 | −1.357 | 0.412 | 4.96E−16 |
| SCAMP3 | O14828 | −1.386 | 0.372 | 5.46E−16 |
| METAP1D | Q6UB28 | −1.311 | 0.348 | 5.49E−16 |
| CBL | P22681 | −1.332 | 0.457 | 7.97E−16 |
| CRKL | P46109 | −1.317 | 0.288 | 1.08E−15 |
| DECR1 | Q16698 | −1.324 | 0.279 | 1.13E−15 |
| PTPN1 | P18031 | −1.331 | 0.350 | 3.22E−15 |
| IRAK4 | Q9NWZ3 | −1.357 | 0.345 | 3.49E−15 |
| KIF1BP | Q96EK5 | −1.392 | 0.315 | 3.57E−15 |
| LRMP | Q12912 | −1.276 | 0.396 | 3.60E−15 |
| VPS53 | Q5VIR6 | −1.391 | 0.461 | 6.81E−15 |
| NAA10 | P41227 | −1.352 | 0.362 | 8.18E−15 |
| SPRY2 | O43597 | −1.316 | 0.445 | 1.03E−14 |
| DCTN1 | Q14203 | −1.243 | 0.396 | 2.45E−14 |
| MANF | P55145 | −1.398 | 0.302 | 3.05E−14 |
| CETN2 | P41208 | −1.215 | 0.599 | 1.50E−13 |
| MYO9B | Q13459 | −1.252 | 0.497 | 4.77E−13 |
| MGMT | P16455 | −1.289 | 0.344 | 8.03E−13 |
| PRDX5 | P30044 | −1.230 | 0.412 | 3.58E−12 |
| NT5C3A | Q9H0P0 | −1.265 | 0.313 | 4.02E−12 |
| PRKCQ | Q04759 | −1.123 | 0.761 | 9.09E−12 |
| VPS37A | Q8NEZ2 | −1.151 | 0.522 | 1.17E−11 |
| HCLS1 | P14317 | −1.304 | 0.378 | 2.25E−11 |
| PVALB | P20472 | −1.235 | 0.262 | 6.59E−11 |
| GAMT | Q14353 | −1.117 | 0.904 | 6.75E−11 |
| STX8 | Q9UNK0 | −1.133 | 0.497 | 3.98E−10 |
| TMSB10 | P63313 | −0.817 | 0.892 | 2.02E−06 |
| PRDX1 | Q06830 | −0.746 | 0.834 | 3.14E−06 |
| GSAP | A4D1B5 | −0.928 | 0.958 | 4.06E−06 |
| VAMP5 | O95183 | −0.785 | 0.940 | 9.83E−06 |
| CD164 | Q04900 | −0.722 | 0.954 | 8.02E−05 |
| LGMN | Q99538 | −0.643 | 0.926 | 2.19E−04 |
| hK14 | Q9P0G3 | 0.530 | 1.220 | 3.08E−03 |
| NELL1 | Q92832 | −0.338 | 0.850 | 2.84E−02 |

TABLE 2
List of 429 plasma proteins associated with AD phenotypes. β, effect size.

| Protein name | Uniprot ID | β | Fold Change | P-value |
|---|---|---|---|---|
| LYN | P07948 | −1.481 | 0.444 | 2.82E−21 |
| CD69 | Q07108 | −1.531 | 0.369 | 5.22E−21 |
| EIF4G1 | Q04637 | −1.396 | 0.257 | 5.44E−21 |
| PLXNA4 | Q9HCM2 | −1.476 | 0.286 | 1.10E−20 |
| SNAP29 | O95721 | −1.397 | 0.357 | 3.61E−20 |
| BCR | P11274 | −1.468 | 0.329 | 7.57E−20 |
| PPP1R9B | Q96SB3 | −1.426 | 0.280 | 7.61E−20 |
| ICA1 | Q05084 | −1.302 | 0.629 | 7.61E−20 |
| TXLNA | P40222 | −1.491 | 0.353 | 9.90E−20 |
| BANK1 | Q8NDB2 | −1.416 | 0.189 | 1.01E−19 |
| ARHGEF12 | Q9NZN5 | −1.420 | 0.244 | 1.70E−19 |
| AXIN1 | O15169 | −1.407 | 0.291 | 2.24E−19 |
| INPPL1 | O15357 | −1.458 | 0.209 | 3.83E−19 |
| CLIP2 | Q9UDT6 | −1.470 | 0.198 | 7.51E−19 |
| CASP-3 | P42574 | −1.358 | 0.248 | 9.24E−19 |
| TDRKH | Q9Y2W6 | −1.424 | 0.322 | 1.01E−18 |
| NEMO | Q9Y6K9 | −1.390 | 0.325 | 1.30E−18 |
| MESDC2 | Q14696 | −1.453 | 0.376 | 1.51E−18 |
| STK4 | Q13043 | −1.395 | 0.216 | 1.65E−18 |
| ITGB1BP2 | Q9UKP3 | −1.469 | 0.300 | 1.65E−18 |
| CALCOCO1 | Q9P1Z2 | −1.369 | 0.216 | 1.94E−18 |
| SRPK2 | P78362 | −1.426 | 0.484 | 2.11E−18 |
| DAPP1 | Q9UN19 | −1.405 | 0.174 | 2.14E−18 |
| DAB2 | P98082 | −1.368 | 0.389 | 2.23E−18 |
| ZBTB16 | Q05516 | −1.442 | 0.475 | 2.90E−18 |
| GRAP2 | O75791 | −1.438 | 0.252 | 2.92E−18 |
| SRC | P12931 | −1.458 | 0.208 | 4.82E−18 |
| SNAP23 | O00161 | −1.369 | 0.224 | 4.85E−18 |
| MAP4K5 | Q9Y4K4 | −1.463 | 0.181 | 5.14E−18 |
| ERBB2IP | Q96RT1 | −1.394 | 0.304 | 8.00E−18 |
| YES1 | P07947 | −1.436 | 0.237 | 8.69E−18 |
| BACH1 | O14867 | −1.407 | 0.535 | 8.86E−18 |
| SH2B3 | Q9UQQ2 | −1.422 | 0.273 | 1.04E−17 |
| FKBP1B | P68106 | −1.381 | 0.398 | 1.11E−17 |
| WASF1 | Q92558 | −1.442 | 0.320 | 1.17E−17 |
| AIFM1 | O95831 | −1.330 | 0.371 | 1.21E−17 |
| MAP2K6 | P52564 | −1.373 | 0.448 | 1.23E−17 |
| TRIM5 | Q9C035 | −1.374 | 0.556 | 1.26E−17 |
| PRTFDC1 | Q9NRG1 | −1.393 | 0.246 | 1.39E−17 |
| CDKN1A | P38936 | −1.410 | 0.287 | 1.56E−17 |
| PMVK | Q15126 | −1.443 | 0.203 | 1.70E−17 |
| FOXO1 | Q12778 | −1.453 | 0.385 | 2.52E−17 |
| USO1 | O60763 | −1.418 | 0.270 | 3.11E−17 |
| HEXIM1 | O94992 | −1.331 | 0.428 | 5.64E−17 |
| GOPC | Q9HD26 | −1.480 | 0.284 | 5.65E−17 |
| AIMP1 | Q12904 | −1.438 | 0.301 | 6.95E−17 |
| TBCB | Q99426 | −1.374 | 0.236 | 8.61E−17 |
| CA13 | Q8N1Q1 | −1.383 | 0.280 | 1.24E−16 |
| TANK | Q92844 | −1.268 | 0.534 | 2.08E−16 |
| TACC3 | Q9Y6A5 | −1.362 | 0.416 | 4.38E−16 |
| NFATC1 | O95644 | −1.383 | 0.435 | 4.90E−16 |
| LAT2 | Q9GZY6 | −1.357 | 0.412 | 4.96E−16 |
| SCAMP3 | O14828 | −1.386 | 0.372 | 5.46E−16 |
| METAP1D | Q6UB28 | −1.311 | 0.348 | 5.49E−16 |
| CBL | P22681 | −1.332 | 0.457 | 7.97E−16 |
| STX6 | O43752 | −1.266 | 0.627 | 9.46E−16 |
| CRKL | P46109 | −1.317 | 0.288 | 1.08E−15 |
| DECR1 | Q16698 | −1.324 | 0.279 | 1.13E−15 |
| SMAD1 | Q15797 | −1.423 | 0.508 | 2.19E−15 |
| IRAK1 | P51617 | −1.291 | 0.594 | 2.39E−15 |
| FKBP5 | Q13451 | −1.330 | 0.420 | 2.59E−15 |
| PTPN1 | P18031 | −1.331 | 0.350 | 3.22E−15 |
| IRAK4 | Q9NWZ3 | −1.357 | 0.345 | 3.49E−15 |
| KIF1BP | Q96EK5 | −1.392 | 0.315 | 3.57E−15 |
| LRMP | Q12912 | −1.276 | 0.396 | 3.60E−15 |
| VPS53 | Q5VIR6 | −1.391 | 0.461 | 6.81E−15 |
| PLA2G4A | P47712 | −1.222 | 0.593 | 7.32E−15 |
| HSP27 | P04792 | −1.296 | 0.519 | 7.38E−15 |
| PPP1R2 | P41236 | −1.357 | 0.556 | 7.86E−15 |
| NAA10 | P41227 | −1.352 | 0.362 | 8.18E−15 |
| STX16 | O14662 | −1.312 | 0.567 | 9.94E−15 |
| SPRY2 | O43597 | −1.316 | 0.445 | 1.03E−14 |
| EGF | P01133 | −1.373 | 0.285 | 1.94E−14 |
| DCTN1 | Q14203 | −1.243 | 0.396 | 2.45E−14 |
| ABL1 | P00519 | −1.264 | 0.688 | 2.86E−14 |
| MANF | P55145 | −1.398 | 0.302 | 3.05E−14 |
| PTPN6 | P29350 | −1.321 | 0.643 | 3.65E−14 |
| FLI1 | Q01543 | −1.296 | 0.534 | 3.70E−14 |
| DRG2 | P55039 | −1.284 | 0.646 | 6.62E−14 |
| GP6 | Q9HCN6 | −1.227 | 0.671 | 7.94E−14 |
| CETN2 | P41208 | −1.215 | 0.599 | 1.50E−13 |
| FGF2 | P09038 | −1.292 | 0.605 | 1.89E−13 |
| LAT | O43561 | −1.291 | 0.330 | 2.03E−13 |
| PPIB | P23284 | −1.307 | 0.626 | 2.17E−13 |
| JAM-A | Q9Y624 | −1.163 | 0.622 | 2.60E−13 |
| YTHDF3 | Q7Z739 | −1.227 | 0.646 | 3.23E−13 |
| MYO9B | Q13459 | −1.252 | 0.497 | 4.77E−13 |
| NUB1 | Q9Y5A7 | −1.240 | 0.529 | 6.50E−13 |
| MGMT | P16455 | −1.289 | 0.344 | 8.03E−13 |
| GFER | P55789 | −1.284 | 0.637 | 1.12E−12 |
| FOXO3 | O43524 | −1.182 | 0.588 | 1.76E−12 |
| PECAM-1 | P16284 | −1.092 | 0.779 | 1.99E−12 |
| CD2AP | Q9Y5K6 | −1.091 | 0.397 | 3.34E−12 |
| PRDX5 | P30044 | −1.230 | 0.412 | 3.58E−12 |
| NT5C3A | Q9H0P0 | −1.265 | 0.313 | 4.02E−12 |
| PRKCQ | Q04759 | −1.123 | 0.761 | 9.09E−12 |
| VPS37A | Q8NEZ2 | −1.151 | 0.522 | 1.17E−11 |
| PRDX3 | P30048 | −1.142 | 0.686 | 1.21E−11 |
| MAX | P61244 | −1.283 | 0.643 | 1.34E−11 |
| ENO2 | P09104 | −1.163 | 0.630 | 1.64E−11 |
| WWP2 | O00308 | −1.112 | 0.655 | 1.66E−11 |
| COL4A3BP | Q9Y5P4 | −1.133 | 0.642 | 1.67E−11 |
| NF2 | P35240 | −1.219 | 0.614 | 1.92E−11 |
| LACTB2 | Q53H82 | −1.215 | 0.522 | 2.14E−11 |
| HCLS1 | P14317 | −1.304 | 0.378 | 2.25E−11 |
| FXYD5 | Q96DB9 | −1.063 | 0.794 | 3.10E−11 |
| CASP2 | P42575 | −1.270 | 0.490 | 3.81E−11 |
| LAP3 | P28838 | −1.071 | 0.760 | 3.86E−11 |
| TOP2B | Q02880 | −1.266 | 0.521 | 3.92E−11 |
| ANXA11 | P50995 | −1.172 | 0.580 | 4.07E−11 |
| ARHGAP25 | P42331 | −1.151 | 0.720 | 5.03E−11 |
| SERPINB6 | P35237 | −1.105 | 0.762 | 6.44E−11 |
| PVALB | P20472 | −1.235 | 0.262 | 6.59E−11 |
| GAME | Q14353 | −1.117 | 0.904 | 6.75E−11 |
| PTPRJ | Q12913 | −1.211 | 0.513 | 7.45E−11 |
| ARHGAP1 | Q07960 | −1.105 | 0.628 | 9.28E−11 |
| TBL1X | O60907 | −1.131 | 0.601 | 9.29E−11 |
| AKR1B1 | P15121 | −1.024 | 0.883 | 9.80E−11 |
| FES | P07332 | −1.186 | 0.640 | 1.05E−10 |
| PLXNB3 | Q9ULL4 | −1.164 | 0.743 | 1.24E−10 |
| BAG6 | P46379 | −1.030 | 0.769 | 1.68E−10 |
| NFKBIE | O00221 | −1.171 | 0.550 | 1.87E−10 |
| ST1A1 | P50225 | −1.048 | 0.565 | 1.93E−10 |
| COMT | P21964 | −1.036 | 0.616 | 2.13E−10 |
| CDC27 | P30260 | −1.148 | 0.657 | 2.39E−10 |
| ILKAP | Q9H0C8 | −1.034 | 0.734 | 3.77E−10 |
| STX8 | Q9UNK0 | −1.133 | 0.497 | 3.98E−10 |
| RRM2B | Q7LG56 | −1.145 | 0.881 | 4.08E−10 |
| HTRA2 | O43464 | −1.092 | 0.832 | 4.10E−10 |
| AKT1S1 | Q96B36 | −1.072 | 0.592 | 4.82E−10 |
| VASH1 | Q7L8A9 | −1.255 | 0.705 | 5.00E−10 |
| TRAF2 | Q12933 | −0.994 | 0.691 | 5.93E−10 |
| BIRC2 | Q13490 | −1.120 | 0.878 | 7.17E−10 |
| EIF4B | P23588 | −1.020 | 0.529 | 1.04E−09 |
| IQGAP2 | Q13576 | −1.061 | 0.907 | 1.04E−09 |
| FADD | Q13158 | −1.089 | 0.657 | 1.28E−09 |
| HMOX2 | P30519 | −1.004 | 0.733 | 1.28E−09 |
| RP2 | O75695 | −0.960 | 0.758 | 1.75E−09 |
| RPS6KB1 | P23443 | −1.133 | 0.781 | 2.10E−09 |
| IMPA1 | P29218 | −1.022 | 0.760 | 3.08E−09 |
| MetAP 2 | P50579 | −1.043 | 0.574 | 3.84E−09 |
| Gal-8 | O00214 | −1.068 | 0.685 | 4.69E−09 |
| WAS | P42768 | −1.040 | 0.541 | 5.50E−09 |
| CRADD | P78560 | −1.043 | 0.520 | 8.13E−09 |
| DCTN2 | Q13561 | −1.025 | 0.729 | 8.57E−09 |
| DFFA | O00273 | −1.048 | 0.697 | 8.66E−09 |
| SELP | P16109 | −0.996 | 0.689 | 9.86E−09 |
| SIRT2 | Q8IXJ6 | −1.009 | 0.458 | 1.20E−08 |
| CD63 | P08962 | −0.906 | 0.749 | 1.24E−08 |
| STAMBP | O95630 | −0.975 | 0.565 | 1.32E−08 |
| TYMP | P19971 | −1.047 | 0.654 | 1.34E−08 |
| DAG1 | Q14118 | −1.066 | 0.871 | 1.43E−08 |
| DIABLO | Q9NR28 | −0.968 | 0.619 | 3.05E−08 |
| STXBP3 | O00186 | −1.102 | 0.775 | 4.60E−08 |
| P4HB | P07237 | −0.937 | 0.811 | 4.75E−08 |
| CD40-L | P29965 | −1.030 | 0.536 | 5.97E−08 |
| NUDT5 | Q9UKK9 | −0.915 | 0.742 | 6.08E−08 |
| PRKRA | O75569 | −1.004 | 0.824 | 7.03E−08 |
| FHIT | P49789 | −0.916 | 0.756 | 7.14E−08 |
| BGN | P21810 | −0.973 | 0.895 | 7.42E−08 |
| TP53 | P04637 | −0.883 | 0.823 | 8.27E−08 |
| PSME1 | Q06323 | −0.873 | 0.757 | 1.61E−07 |
| KYAT1 | Q16773 | −0.982 | 0.610 | 1.74E−07 |
| WASF3 | Q9UPY6 | −1.004 | 0.664 | 1.79E−07 |
| CLEC1B | Q9P126 | −0.867 | 0.664 | 2.35E−07 |
| USP8 | P40818 | −0.973 | 0.648 | 3.50E−07 |
| MIF | P14174 | −0.882 | 0.600 | 3.56E−07 |
| IRF9 | Q00978 | −1.052 | 0.773 | 4.32E−07 |
| PARK7 | Q99497 | −0.847 | 0.696 | 4.77E−07 |
| EDAR | Q9UNE0 | −0.908 | 0.724 | 5.55E−07 |
| DGKZ | Q13574 | −0.941 | 0.919 | 5.58E−07 |
| BTC | P35070 | −0.912 | 0.746 | 6.29E−07 |
| SCARF1 | Q14162 | −0.855 | 0.855 | 7.58E−07 |
| MVK | Q03426 | −0.830 | 0.683 | 9.05E−07 |
| ERP44 | Q9BS26 | −0.827 | 0.845 | 1.02E−06 |
| DNAJB1 | P25685 | −0.845 | 0.583 | 1.03E−06 |
| LIF-R | P42702 | 0.722 | 1.139 | 1.18E−06 |
| ARSB | P15848 | −0.835 | 0.834 | 1.63E−06 |
| MAGED1 | Q9Y5V3 | −0.941 | 0.882 | 1.93E−06 |
| TMSB10 | P63313 | −0.817 | 0.892 | 2.02E−06 |
| ANXA4 | P09525 | −0.937 | 0.847 | 2.84E−06 |
| QDPR | P09417 | −0.823 | 0.725 | 3.03E−06 |
| PRDX1 | Q06830 | −0.746 | 0.834 | 3.14E−06 |
| AHCY | P23526 | −0.688 | 0.889 | 3.31E−06 |
| PRKAB1 | Q9Y478 | −0.884 | 0.852 | 3.81E−06 |
| PAG1 | Q9NWQ8 | −0.749 | 0.782 | 3.86E−06 |
| GSAP | A4D1B5 | −0.928 | 0.958 | 4.06E−06 |
| CCT5 | P48643 | −0.898 | 0.805 | 5.42E−06 |
| STIP1 | P31948 | −0.805 | 0.891 | 6.60E−06 |
| VAMP5 | O95183 | −0.785 | 0.940 | 9.83E−06 |
| HDGF | P51858 | −0.747 | 0.772 | 1.12E−05 |
| KYNU | Q16719 | −0.819 | 0.766 | 1.35E−05 |
| INPP1 | P49441 | −0.753 | 0.850 | 1.45E−05 |
| GLB1 | P16278 | −0.696 | 0.852 | 1.69E−05 |
| ACAA1 | P09110 | −0.712 | 0.691 | 1.77E−05 |
| MCFD2 | Q8NI22 | −0.732 | 0.902 | 1.89E−05 |
| PAK4 | O96013 | −1.029 | 0.853 | 2.60E−05 |
| ENAH | Q8N8S7 | −0.739 | 0.822 | 3.34E−05 |
| SH2D1A | O60880 | −0.720 | 0.903 | 3.56E−05 |
| FKBP7 | Q9Y680 | −0.717 | 0.747 | 4.07E−05 |
| PLXDC1 | Q8IUK5 | −0.681 | 0.900 | 4.25E−05 |
| TXNDC5 | Q8NBS9 | −0.695 | 0.908 | 4.63E−05 |
| BID | P55957 | −0.762 | 0.758 | 4.64E−05 |
| MAEA | Q7L5Y9 | −0.689 | 0.769 | 5.20E−05 |
| CXCL1 | P09341 | −0.740 | 0.775 | 5.38E−05 |
| PAR-1 | P25116 | −0.707 | 0.884 | 5.82E−05 |
| CCL5 | P13501 | −0.640 | 0.506 | 5.91E−05 |
| ITGB1BP1 | O14713 | −0.652 | 1.248 | 6.27E−05 |
| EGLN1 | Q9GZT9 | −0.624 | 0.984 | 6.90E−05 |
| CD164 | Q04900 | −0.722 | 0.954 | 8.02E−05 |
| TIGAR | Q9NQ88 | −0.720 | 1.036 | 8.20E−05 |
| ATP6V1D | Q9Y5K8 | −0.647 | 1.010 | 9.59E−05 |
| AIF1 | P55008 | −0.733 | 0.453 | 1.01E−04 |
| RASSF2 | P50749 | −0.675 | 0.877 | 1.26E−04 |
| EIF5A | P63241 | −0.653 | 0.932 | 1.32E−04 |
| PEBP1 | P30086 | −0.666 | 0.822 | 1.36E−04 |
| DPP7 | Q9UHL4 | −0.677 | 0.815 | 1.63E−04 |
| PPM1B | O75688 | −0.695 | 0.933 | 1.96E−04 |
| LGMN | Q99538 | −0.643 | 0.926 | 2.19E−04 |
| GALNT2 | Q10471 | −0.674 | 0.886 | 2.43E−04 |
| FKBP4 | Q02790 | −0.761 | 0.798 | 2.78E−04 |
| CD84 | Q9UIB8 | −0.670 | 0.881 | 2.83E−04 |
| PIK3AP1 | Q6ZUJ8 | −0.601 | 0.792 | 2.91E−04 |
| PRDX6 | P30041 | −0.695 | 0.818 | 2.92E−04 |
| CNTN5 | O94779 | −0.582 | 0.925 | 3.04E−04 |
| GPIBA | P07359 | −0.722 | 0.727 | 3.37E−04 |
| ITGA6 | P23229 | −0.696 | 0.775 | 3.53E−04 |
| NAMPT | P43490 | −0.642 | 0.827 | 3.87E−04 |
| ATG4A | Q8WYN0 | −0.579 | 0.820 | 3.88E−04 |
| PFDN2 | Q9UHV9 | −0.634 | 0.922 | 4.32E−04 |
| CALR | P27797 | −0.699 | 0.877 | 4.66E−04 |
| DDX58 | O95786 | −0.672 | 0.812 | 4.68E−04 |
| CD40 | P25942 | −0.619 | 0.939 | 5.06E−04 |
| SUMF2 | Q8NBJ7 | −0.577 | 0.788 | 5.09E−04 |
| BLM hydrolase | Q13867 | −0.584 | 0.605 | 5.82E−04 |
| CAMKK1 | Q8N5S9 | −0.665 | 0.901 | 6.83E−04 |
| KLK4 | Q9Y5K2 | 0.457 | 1.966 | 7.05E−04 |
| CXCL5 | P42830 | −0.573 | 0.790 | 7.52E−04 |
| TCL1A | P56279 | −0.624 | 0.520 | 8.27E−04 |
| PFKM | P08237 | −0.543 | 0.849 | 8.60E−04 |
| FGR | P09769 | −0.621 | 0.898 | 9.47E−04 |
| TPP1 | O14773 | −0.596 | 0.925 | 9.75E−04 |
| STC1 | P52823 | 0.652 | 1.171 | 1.07E−03 |
| NUCB2 | P80303 | −0.649 | 0.928 | 1.13E−03 |
| LAMA4 | Q16363 | −0.566 | 0.993 | 1.15E−03 |
| TRIM21 | P19474 | −0.846 | 0.701 | 1.24E−03 |
| ING1 | Q9UK53 | −0.580 | 0.946 | 1.26E−03 |
| PTX3 | P26022 | 0.590 | 1.154 | 1.38E−03 |
| PPP3R1 | P63098 | −0.610 | 0.911 | 1.39E−03 |
| ABHD14B | Q96IU4 | −0.709 | 0.881 | 1.40E−03 |
| EGFR | P00533 | −0.508 | 0.937 | 1.43E−03 |
| MMP7 | P09237 | 0.467 | 1.209 | 1.48E−03 |
| MEP1B | Q16820 | −0.509 | 0.787 | 1.58E−03 |
| ITGB7 | P26010 | −0.559 | 0.961 | 1.62E−03 |
| LRP1 | Q07954 | −0.586 | 0.921 | 1.69E−03 |
| AOC3 | Q16853 | −0.531 | 0.963 | 1.71E−03 |
| CD8A | P01732 | 0.509 | 1.201 | 1.82E−03 |
| ATP6V1F | Q16864 | −0.554 | 0.946 | 1.94E−03 |
| NADK | O95544 | −0.528 | 0.913 | 1.99E−03 |
| PTP4A1 | Q93096 | −0.520 | 1.051 | 2.10E−03 |
| IL1B | P01584 | −0.546 | 0.993 | 2.10E−03 |
| HSPB6 | O14558 | 0.485 | 1.226 | 2.16E−03 |
| SKAP1 | Q86WV1 | −0.570 | 0.769 | 2.18E−03 |
| HPGDS | O60760 | −0.512 | 0.902 | 2.30E−03 |
| SPINK4 | O60575 | 0.514 | 1.441 | 2.37E−03 |
| CNPY2 | Q9Y2B0 | −0.541 | 0.894 | 2.39E−03 |
| CD46 | P15529 | −0.547 | 0.892 | 2.66E−03 |
| IGSF3 | O75054 | −0.460 | 0.828 | 2.76E−03 |
| uPA | P00749 | −0.481 | 0.877 | 2.83E−03 |
| Dkk-4 | Q9UBT3 | 0.496 | 1.959 | 3.00E 03 |
| CRELD2 | Q6UXH1 | −0.498 | 0.934 | 3.03E−03 |
| FAP | Q12884 | −0.532 | 0.917 | 3.07E−03 |
| hK14 | Q9POG3 | 0.530 | 1.220 | 3.08E−03 |
| CD97 | P48960 | −0.509 | 0.890 | 3.37E−03 |
| RET | P07949 | −0.454 | 0.841 | 3.59E−03 |
| FETUB | Q9UGM5 | −0.550 | 0.919 | 3.61E−03 |
| TNFSF13B | Q9Y275 | −0.494 | 0.981 | 3.76E−03 |
| PAPPA | Q13219 | 0.558 | 1.173 | 4.03E−03 |
| CSF-1 | P09603 | 0.500 | 1.075 | 4.13E−03 |
| THOP1 | P52888 | −0.521 | 0.874 | 4.13E−03 |
| ITGB1 | P05556 | −0.481 | 0.954 | 4.19E−03 |
| KRT19 | P08727 | 0.536 | 1.230 | 4.25E−03 |
| GLO1 | Q04760 | −0.450 | 0.850 | 4.34E−03 |
| SOD2 | P04179 | −0.552 | 0.966 | 4.51E−03 |
| PAI | P05121 | −0.485 | 0.790 | 4.68E−03 |
| MMP-3 | P08254 | 0.405 | 1.182 | 4.76E−03 |
| ALDH1A1 | P00352 | −0.422 | 0.823 | 4.77E−03 |
| FGF-5 | P12034 | 0.432 | 1.143 | 5.40E−03 |
| TNFAIP8 | O95379 | −0.532 | 0.934 | 5.44E−03 |
| PDP1 | Q9P0J1 | −0.496 | 0.956 | 5.98E−03 |
| SMOC1 | Q9H4F8 | 0.480 | 1.136 | 6.05E−03 |
| GUSB | P08236 | −0.503 | 0.721 | 6.07E−03 |
| DPP10 | Q8N608 | −0.461 | 0.996 | 6.41E−03 |
| AGRP | O00253 | 0.507 | 1.069 | 6.48E−03 |
| PSIP1 | O75475 | −0.458 | 0.822 | 6.55E−03 |
| ITGB2 | P05107 | −0.442 | 0.875 | 6.78E−03 |
| FUT8 | Q9BYC5 | −0.478 | 0.863 | 6.86E−03 |
| DEFB4A | O15263 | 0.464 | 1.441 | 7.03E−03 |
| MASP1 | P48740 | −0.406 | 0.956 | 7.24E−03 |
| SIRT5 | Q9NXA8 | −0.486 | 0.945 | 7.38E−03 |
| CX3CL1 | P78423 | 0.475 | 1.230 | 7.52E−03 |
| APBB1IP | Q7Z5R6 | −0.478 | 0.973 | 7.61E−03 |
| ENTPD2 | Q9Y5L3 | −0.438 | 0.938 | 8.26E−03 |
| DCTPP1 | Q9H773 | −0.491 | 0.923 | 8.42E−03 |
| CSNK1D | P48730 | −0.528 | 1.152 | 8.43E−03 |
| SDC4 | P31431 | −0.481 | 0.730 | 8.72E−03 |
| AARSD1 | Q9BTE6 | −0.444 | 0.897 | 8.87E−03 |
| CRHBP | P24387 | −0.414 | 0.928 | 9.04E−03 |
| ITGA11 | Q9UKX5 | −0.423 | 0.874 | 9.29E−03 |
| PHOSPHO1 | Q8TCT1 | 0.467 | 1.123 | 9.80E−03 |
| TNC | P24821 | 0.456 | 1.183 | 1.01E−02 |
| CFC1 | P0CG37 | 0.423 | 1.187 | 1.01E−02 |
| CNTN2 | Q02246 | −0.430 | 0.957 | 1.03E−02 |
| SYND1 | P18827 | −0.484 | 0.943 | 1.03E−02 |
| HB-EGF | Q99075 | −0.451 | 0.833 | 1.04E−02 |
| TGF-alpha | P01135 | 0.431 | 1.133 | 1.08E−02 |
| CTRC | Q99895 | 0.474 | 1.254 | 1.09E−02 |
| WNT9A | O14904 | 0.455 | 1.228 | 1.11E−02 |
| CCL17 | Q92583 | −0.466 | 0.851 | 1.11E−02 |
| C1QA | P02745 | 0.487 | 1.124 | 1.13E−02 |
| BRK1 | Q8WUW1 | −0.444 | 0.958 | 1.14E−02 |
| NCS1 | P62166 | 0.402 | 1.105 | 1.17E−02 |
| ANXA1 | P04083 | −0.518 | 0.973 | 1.19E−02 |
| LTA4H | P09960 | −0.489 | 0.968 | 1.19E−02 |
| CDHR5 | Q9HBB8 | −0.395 | 0.886 | 1.21E−02 |
| NRTN | Q99748 | −0.410 | 1.355 | 1.22E−02 |
| SEPT9 | Q9UHD8 | −0.501 | 0.972 | 1.25E−02 |
| DPEP1 | P16444 | 0.437 | 1.096 | 1.25E−02 |
| CTF1 | Q16619 | −0.439 | 0.955 | 1.26E−02 |
| CCL11 | P51671 | 0.367 | 1.155 | 1.28E−02 |
| GALNT10 | Q86SR1 | −0.507 | 0.923 | 1.31E−02 |
| ROBO2 | Q9HCK4 | −0.449 | 0.976 | 1.37E−02 |
| FAM3B | P58499 | 0.450 | 1.177 | 1.45E−02 |
| CHL1 | O00533 | 0.457 | 1.050 | 1.46E−02 |
| DDC | P20711 | −0.463 | 0.914 | 1.46E−02 |
| MCP-1 | P13500 | −0.434 | 1.167 | 1.46E−02 |
| IL13RA1 | P78552 | −0.405 | 0.932 | 1.48E−02 |
| FGF-BP1 | Q14512 | 0.390 | 1.080 | 1.48E−02 |
| PCSK9 | Q8NBP7 | −0.387 | 0.968 | 1.53E−02 |
| OSMR | Q99650 | 0.460 | 1.050 | 1.56E−02 |
| IL7 | P13232 | −0.407 | 0.962 | 1.57E−02 |
| ALCAM | Q13740 | −0.389 | 1.006 | 1.57E−02 |
| CDON | Q4KMG0 | −0.451 | 0.951 | 1.64E−02 |
| SIGLEC7 | Q9Y286 | −0.453 | 0.942 | 1.65E−02 |
| PDGF subunit A | P04085 | −0.399 | 0.866 | 1.66E−02 |
| IFNLR1 | Q8IU57 | −0.444 | 0.901 | 1.73E−02 |
| CDH17 | Q12864 | −0.441 | 0.908 | 1.86E−02 |
| TR-AP | P13686 | −0.431 | 0.940 | 1.94E−02 |
| DPP4 | P27487 | −0.395 | 0.904 | 1.99E−02 |
| 4E-BP1 | Q13541 | −0.397 | 0.902 | 2.06E−02 |
| PARP-1 | P09874 | −0.467 | 0.865 | 2.08E−02 |
| IL-1RT2 | P27930 | −0.399 | 0.933 | 2.11E−02 |
| TRAIL | P50591 | −0.403 | 0.938 | 2.15E−02 |
| NCF2 | P19878 | −0.422 | 0.886 | 2.15E−02 |
| TNFSF14 | 043557 | −0.448 | 0.903 | 2.16E−02 |
| FLT1 | P17948 | 0.365 | 1.087 | 2.16E−02 |
| XCL1 | P47992 | 0.366 | 1.234 | 2.18E−02 |
| TNFRSF14 | Q92956 | −0.350 | 1.050 | 2.26E−02 |
| SCG2 | P13521 | 0.380 | 1.130 | 2.28E−02 |
| CHIT1 | Q13231 | 0.413 | 1.358 | 2.29E−02 |
| PXN | P49023 | −0.376 | 0.958 | 2.29E−02 |
| CES2 | O00748 | −0.429 | 0.911 | 2.32E−02 |
| VCAM1 | P19320 | 0.402 | 1.090 | 2.32E−02 |
| BAMB1 | Q13145 | 0.413 | 1.106 | 2.33E−02 |
| SOD1 | P00441 | −0.433 | 0.809 | 2.35E−02 |
| CYR61 | O00622 | 0.386 | 1.235 | 2.38E−02 |
| NBN | O60934 | −0.504 | 0.937 | 2.40E−02 |
| VAT1 | Q99536 | −0.397 | 0.936 | 2.44E−02 |
| EZR | P15311 | −0.432 | 0.970 | 2.51E−02 |
| ERBB2 | P04626 | −0.351 | 0.942 | 2.52E−02 |
| ACTN4 | O43707 | −0.405 | 1.158 | 2.55E−02 |
| COCH | O43405 | −0.387 | 0.924 | 2.59E−02 |
| FUS | P35637 | −0.438 | 0.894 | 2.60E−02 |
| DCN | P07585 | 0.419 | 1.104 | 2.67E−02 |
| ESAM | Q96AP7 | −0.344 | 1.006 | 2.67E−02 |
| NFATC3 | Q12968 | −0.399 | 0.537 | 2.78E−02 |
| APEX1 | P27695 | −0.428 | 0.932 | 2.81E−02 |
| NELL1 | Q92832 | −0.338 | 0.850 | 2.84E−02 |
| TRAIL-R2 | O14763 | 0.349 | 1.187 | 2.87E−02 |
| PRSS2 | P07478 | 0.368 | 1.189 | 2.90E−02 |
| ERBB3 | P21860 | −0.393 | 0.963 | 2.90E−02 |
| METAP1 | P53582 | −0.447 | 0.899 | 2.97E−02 |
| PPY | P01298 | 0.338 | 1.416 | 3.01E−02 |
| CBLN4 | Q9NTU7 | −0.405 | 0.890 | 3.04E−02 |
| UMOD | P07911 | −0.336 | 0.948 | 3.04E−02 |
| HNMT | P50135 | −0.377 | 0.990 | 3.06E−02 |
| MMP-1 | P03956 | −0.368 | 0.893 | 3.07E−02 |
| CNDP1 | Q96KN2 | −0.322 | 0.881 | 3.17E−02 |
| SNCG | O76070 | 0.350 | 1.228 | 3.19E−02 |
| CTSD | PO7339 | −0.374 | 0.866 | 3.21E−02 |
| SCLY | Q96I15 | −0.432 | 0.829 | 3.25E−02 |
| PDGF-R-alpha | P16234 | 0.403 | 1.107 | 3.30E−02 |
| MIC-A/B | Q29983, Q29980 | −0.378 | 0.890 | 3.46E−02 |
| ADM | P35318 | 0.372 | 1.164 | 3.52E−02 |
| OMG | P23515 | −0.396 | 0.841 | 3.53E−02 |
| TIMP4 | Q99727 | 0.376 | 1.356 | 3.57E−02 |
| CANT1 | Q8WVQ1 | −0.349 | 0.985 | 3.60E−02 |
| ANGPTL4 | Q9BY76 | 0.388 | 1.145 | 3.62E−02 |
| AREG | P15514 | 0.328 | 1.138 | 3.62E−02 |
| NOMO1 | Q15155 | −0.340 | 0.900 | 3.65E−02 |
| CDH5 | P33151 | −0.346 | 0.967 | 3.71E−02 |
| S100A11 | P31949 | −0.373 | 0.994 | 3.78E−02 |
| FAS | P25445 | −0.337 | 1.000 | 3.89E−02 |
| TNFRSF10A | O00220 | 0.374 | 1.202 | 3.97E−02 |
| CPM | P14384 | −0.382 | 0.970 | 3.98E−02 |
| VEGFD | O43915 | −0.361 | 0.982 | 3.99E−02 |
| AOC1 | P19801 | −0.352 | 0.992 | 4.00E−02 |
| FLT3 | P36888 | 0.399 | 1.027 | 4.02E−02 |
| FABP9 | Q0Z7S8 | −0.333 | 0.885 | 4.07E−02 |
| MANSC1 | Q9H8J5 | 0.453 | 1.080 | 4.08E−02 |
| PLA2G10 | O15496 | 0.387 | 1.310 | 4.20E−02 |
| GFR-alpha-1 | P56159 | 0.288 | 1.221 | 4.27E−02 |
| PDGF subunit B | P01127 | −0.344 | 0.868 | 4.35E−02 |
| EPHA10 | Q5JZY3 | −0.355 | 1.107 | 4.40E−02 |
| IGFBP3 | P17936 | −0.338 | 0.916 | 4.50E−02 |
| IGFBP-2 | P18065 | 0.318 | 1.313 | 4.53E−02 |
| TGFBR3 | Q03167 | 0.372 | 1.093 | 4.61E−02 |
| FBP1 | P09467 | −0.372 | 0.963 | 4.61E−02 |
| CLSTN2 | Q9H4D0 | 0.316 | 1.107 | 4.62E−02 |
| FGF-19 | O95750 | 0.384 | 1.302 | 4.62E−02 |
| PAM | P19021 | −0.372 | 0.976 | 4.65E−02 |
| CLSPN | Q9HAW4 | −0.362 | 0.908 | 4.71E−02 |
| TR | P02786 | 0.388 | 1.221 | 4.72E−02 |
| N2DL-2 | Q9BZM5 | 0.336 | 1.235 | 4.79E−02 |
| TN-R | Q92752 | −0.383 | 0.891 | 4.83E−02 |
| LYPD1 | Q8N2G4 | −0.389 | 0.912 | 4.87E−02 |
| CNTN1 | Q12860 | −0.292 | 1.012 | 4.88E−02 |
| PREB | Q9HCU5 | −0.420 | 1.003 | 4.89E−02 |
| ZBTB17 | Q13105 | −0.342 | 0.927 | 4.94E−02 |

TABLE 3
List of 12 plasma proteins used for AD risk prediction and
evaluation. β, effect size.

| Protein name | Uniprot ID | β | Fold Change | P-value |
|---|---|---|---|---|
| CETN2 | P41208 | −1.215 | 0.599 | 1.50E−13 |
| PRKCQ | Q04759 | −1.123 | 0.761 | 9.09E−12 |
| VPS37A | Q8NEZ2 | −1.151 | 0.522 | 1.17E−11 |
| GAMT | Q14353 | −1.117 | 0.904 | 6.75E−11 |
| TMSB10 | P63313 | −0.817 | 0.892 | 2.02E−06 |
| PRDX1 | Q06830 | −0.746 | 0.834 | 3.14E−06 |
| GSAP | A4D1B5 | −0.928 | 0.958 | 4.06E−06 |
| VAMP5 | O95183 | −0.785 | 0.940 | 9.83E−06 |
| CD164 | Q04900 | −0.722 | 0.954 | 8.02E−05 |
| LGMN | Q99538 | −0.643 | 0.926 | 2.19E−04 |
| hK14 | Q9P0G3 | 0.530 | 1.220 | 3.08E−03 |
| NELL1 | Q92832 | −0.338 | 0.850 | 2.84E−02 |

TABLE 4
List of 19 plasma proteins used for AD risk prediction and
evaluation. β, effect size.

| Protein name | Uniprot ID | β | Fold Change | P-value |
|---|---|---|---|---|
| LYN | P07948 | −1.481 | 0.444 | 2.82E−21 |
| CASP-3 | P42574 | −1.358 | 0.248 | 9.24E−19 |
| CETN2 | P41208 | −1.215 | 0.599 | 1.50E−13 |
| PRKCQ | Q04759 | −1.123 | 0.761 | 9.09E−12 |
| VPS37A | Q8NEZ2 | −1.151 | 0.522 | 1.17E−11 |
| GAMT | Q14353 | −1.117 | 0.904 | 6.75E−11 |
| NFKBIE | O00221 | −1.171 | 0.550 | 1.87E−10 |
| LIF-R | P42702 | 0.722 | 1.139 | 1.18E−06 |
| TMSB10 | P63313 | −0.817 | 0.892 | 2.02E−06 |
| PRDX1 | Q06830 | −0.746 | 0.834 | 3.14E−06 |
| GSAP | A4D1B5 | −0.928 | 0.958 | 4.06E−06 |
| VAMP5 | O95183 | −0.785 | 0.940 | 9.83E−06 |
| CD 164 | Q04900 | −0.722 | 0.954 | 8.02E−05 |
| LGMN | Q99538 | −0.643 | 0.926 | 2.19E−04 |
| KLK4 | Q9Y5K2 | 0.457 | 1.966 | 7.05E−04 |
| AOC3 | Q16853 | −0.531 | 0.963 | 1.71E−03 |
| CD8A | P01732 | 0.509 | 1.201 | 1.82E−03 |
| hK14 | Q9P0G3 | 0.530 | 1.220 | 3.08E−03 |
| NELL1 | Q92832 | −0.338 | 0.850 | 2.84E−02 |

TABLE 5
Weighted coefficients (β ᵢ) and intercept (ε) for the model
utilizing 12 plasma proteins.

| | 6.642180 | |
|---|---|---|
| Intercept (ε) | Protein name | βᵢ |
| Weighted coefficients (βᵢ) | CETN2 | −1.265698 |
| | PRKCQ | −0.472866 |
| | VPS37A | −0.175694 |
| | GAMT | −0.019014 |
| | TMSB10 | −0.156101 |
| | PRDX1 | −0.321325 |
| | GSAP | 0.004747 |
| | VAMP5 | −0.035239 |
| | CD164 | −0.096450 |
| | LGMN | −0.109538 |
| | hK14 | 0.064363 |
| | NELL1 | −0.004707 |

TABLE 6
Weighted coefficients (βᵢ) and intercept (ε) for the model
utilizing 19 plasma proteins.

| | 5.6563747 | |
|---|---|---|
| Intercept (ε) | Protein name | βᵢ |
| Weighted coefficients (βᵢ) | LYN | −0.3666035 |
| | CASP-3 | 0.0020263 |
| | CETN2 | −0.2037026 |
| | PRKCQ | −0.0633344 |
| | VPS37A | −0.2378607 |
| | GAMT | −0.0165283 |
| | NFKBIE | −0.0105852 |
| | LIF-R | 0.2475330 |
| | TMSB10 | −0.4355160 |
| | PRDX1 | −0.3812860 |
| | GSAP | 0.0010057 |
| | VAMP5 | −0.0418372 |
| | CD164 | −0.5233664 |
| | LGMN | 0.2950641 |
| | KLK4 | 0.0935258 |
| | AOC3 | −0.4224705 |
| | CD8A | 0.0006992 |
| | hK14 | 0.0826993 |
| | NELL1 | −0.0015627 |

TABLE 7
Weighted coefficients (βᵢ) and intercept (ε) for the model utilizing
plasma Aβ_{42/40} ratio, plasma NfL and 12 plasma proteins.

| | 8.384 | |
|---|---|---|
| Intercept (ε) | Protein name | βᵢ |
| Weighted coefficients (βᵢ) | Aβ_{42/40} ratio | −101.2 |
| | NfL | 0.1921 |
| | CETN2 | −1.095 |
| | PRKCQ | −0.6999 |
| | VPS37A | −0.2601 |
| | GAMT | −0.01069 |
| | TMSB10 | −0.3076 |
| | PRDX1 | −0.0529 |
| | GSAP | −0.004979 |
| | VAMP5 | 0.04443 |
| | CD164 | −0.3899 |
| | LGMN | 0.0193 |
| | hK14 | 0.06104 |
| | NELL1 | −0.0002459 |

TABLE 8
Weighted coefficients (βᵢ) and intercept (ε) for the model utilizing
plasma Aβ_{42/40} ratio, plasma NfL and 19 plasma proteins.

| | 12.89 | |
|---|---|---|
| Intercept (ε) | Protein name | βᵢ |
| Weighted coefficients (βᵢ) | Aβ_{42/40} ratio | −163.3 |
| | NfL | 0.1861 |
| | LYN | −0.4666 |
| | CASP-3 | −0.0002276 |
| | CETN2 | 0.04377 |
| | PRKCQ | 0.04734 |
| | VPS37A | −0.2106 |
| | GAMT | −0.1079 |
| | NFKBIE | −0.004808 |
| | LIF-R | 0.4067 |
| | TMSB10 | −0.4735 |
| | PRDX1 | −0.1006 |
| | GSAP | −0.02067 |
| | VAMP5 | 0.08683 |
| | CD164 | −1.068 |
| | LGMN | 0.5571 |
| | KLK4 | 0.05748 |
| | AOC3 | −0.7969 |
| | CD8A | 0.000977 |
| | hK14 | 0.1189 |
| | NELL1 | 0.001718 |

TABLE 9
Weighted coefficients (βᵢ) for plasma Aβ_{42/40} ratio and NfL level.

| | Protein name | βᵢ |
|---|---|---|
| Weighted coefficient (βᵢ) | Aβ_{42/40} ratio | 0.14253 |
| | NfL | −78.84141 |

### REFERENCES

1. Alzheimer's Association. (2016). 2016 Alzheimer's disease facts and figures. Alzheimer's & Dementia, 12(4), 459-509.
2. McKhann, G., Drachman, D., Folstein, M., Katzman, R., Price, D., & Stadlan, E. M. (1984). Clinical diagnosis of Alzheimer's disease: Report of the NINCDS-ADRDA Work Group* under the auspices of Department of Health and Human Services Task Force on Alzheimer's Disease. Neurology, 34(7), 939-939.
3. Carrillo, Maria C., et al. “Revisiting the framework of the National Institute on Aging-Alzheimer's Association diagnostic criteria.” Alzheimer's & Dementia 9.5 (2013): 594-601.
4. Prince, M. J. (2015). World Alzheimer Report 2015: the global impact of dementia: an analysis of prevalence, incidence, cost and trends. Alzheimer's Disease International.
5. Jack Jr, C. R., Bennett, D. A., Blennow, K., Carrillo, M. C., Dunn, B., Haeberlein, S. B., . . . & Liu, E. (2018). NIA-AA research framework: toward a biological definition of Alzheimer's disease. Alzheimer's & Dementia, 14(4), 535-562.
6. Nakamura, A., Kaneko, N., Villemagne, V. L., Kato, T., Doecke, J., Doré, V., . . . & Tomita, T. (2018). High performance plasma amyloid-β biomarkers for Alzheimer's disease. Nature, 554(7691), 249.
7. Preische, O., Schultz, S. A., Apel, A., Kuhle, J., Kaeser, S. A., Barro, C., . . . & Vöglein, J. (2019). Serum neurofilament dynamics predicts neurodegeneration and clinical progression in presymptomatic Alzheimer's disease. Nature medicine, 25(2), 277-283.
8. Religa, P., Cao, R., Religa, D., Xue, Y., Bogdanovic, N., Westaway, D., . . . & Cao, Y. (2013). VEGF significantly restores impaired memory behavior in Alzheimer's mice by improvement of vascular survival. Scientific reports, 3, 2053.
9. American Psychiatric Association. Diagnostic and statistical manual of mental disorders (DSM-5®). (Washington, D C, 2013).
10. Pangman, Verna C., Jeff Sloan, and Lorna Guse. “An examination of psychometric properties of the mini-mental state examination and the standardized mini-mental state examination: implications for clinical practice.” Applied Nursing Research 13.4 (2000): 209-213.
11. Zhou, Xiaopu, et al. “Non-coding variability at the APOE locus contributes to the Alzheimer's risk.” Nature communications 10.1 (2019): 1-16.

1. A method for assessing risk for Alzheimer's Disease (AD) in a subject, comprising:
(1) comparing the subject's plasma or serum or whole blood level of any one protein selected from Tables 1-4 with a standard control level of the same protein found in the plasma or serum or whole blood of an average healthy subject not suffering from or at increased risk for AD;
(2) detecting an increase in the subject's plasma or serum or whole blood level of the protein (which has a positive β value in Table 1, 2, 3, or 4) from the standard control level or detecting a decrease in the subject' plasma or serum or whole blood level of the protein (which has a negative β value in Table 1, 2, 3, or 4) from the standard control level; and
(3) determining the subject as having increased risk for AD.

2. The method of claim 1, wherein the protein is selected from Table 1.

3. The method of claim 2, wherein the protein is selected from Table 3.

4. The method of claim 3, wherein the protein is selected from Table 4.

5. The method of claim 1, further comprising, prior to step (1), measuring the plasma or serum or whole blood level of the protein.

6. The method of claim 5, further comprising, prior to the measuring step, obtaining a plasma or serum or whole blood sample from the subject.

7. A method for assessing risk for Alzheimer's Disease (AD) in two subjects, comprising:
(i) comparing the first subject's plasma or serum or whole blood level of any one protein selected from Tables 1-4 with the second subject's plasma or serum or whole blood level of the same protein;
(ii) detecting the second subject's plasma or serum or whole blood level of the protein higher than the first subject's plasma or serum or whole blood level of the protein (which has a positive β value in Table 1, 2, 3, or 4) or detecting the second subject's plasma or serum or whole blood level of the protein lower than the first subject's plasma or serum or whole blood level of the protein (which has a negative β value in Table 1, 2, 3, or 4); and
(iii) determining the second subject as having a higher risk for AD than the first subject.

8. The method of claim 7, wherein the protein is selected from Table 1.

9. The method of claim 8, wherein the protein is selected from Table 3.

10. The method of claim 9, wherein the protein is selected from Table 4.

11. The method of claim 7, further comprising, prior to step (i), measuring the plasma or serum or whole blood level of the protein.

12. The method of claim 11, further comprising, prior to the measuring step, obtaining a plasma or serum or whole blood sample from the subject.

13. A kit for assessing risk for Alzheimer's Disease (AD) in a subject, comprising a reagent capable of determining the subject's plasma or serum or whole blood level of each of any 5, 10, 15, or 20 proteins independently selected from Table 2.

14-18. (canceled)

19. A detection chip for assessing risk for Alzheimer's Disease (AD) in a subject, comprising a solid substrate and a reagent capable of determining the subject's plasma or serum or whole blood level of each of any 5, 10, 15, or 20 proteins independently selected from Table 2, wherein each reagent is immobilized at an addressable location on the substrate.

20-22. (canceled)

23. A method for assessing risk for Alzheimer's Disease (AD) in a subject, comprising:
(1) calculating a prediction score by inputting a set of values into the formula:
$$Individual AD prediction score = \frac{1}{1 + {e}^{- ( {β}_{i} Candidate {protein}_{i} + ε )}} ,$$
and
(2) determining the subject who has a score from 0 to 0.25±0.05 as having low risk for AD, determining the subject who has a score from above 0.25±0.05 to 0.80±0.01 as having moderate risk for AD, and determining the subject who has a score from above 0.80±0.01 to 1 as having high risk for AD,
wherein the set of values comprises the plasma or serum or whole blood level of each of the 12 proteins set forth in Table 3, and wherein the weighted coefficients (βᵢ) and intercept (ε) of the proteins are set forth in Tables 5-8.

24-29. (canceled)

30. A method for assessing risk for Alzheimer's Disease (AD) among two subjects, comprising:
(i) calculating a prediction score for each of the two subjects by inputting a set of values into the formula:
$$Individual AD prediction score = \frac{1}{1 + {e}^{- ( {β}_{i} Candidate {protein}_{i} )}} ,$$
and
(ii) determining the subject who has a higher score as having an higher risk for AD than the other subject,
wherein the set of values comprises the ratio between the plasma or serum or whole blood levels of amyloid β protein 42 and amyloid β protein 40, the plasma or serum or whole blood level of NfL, the plasma or serum or whole blood level of at least one of the proteins set forth in Table 2, and wherein the corresponding weighted coefficients (βᵢ) are set forth in Table 1, 2, 3, 4, and 9.

31-36. (canceled)

37. A method for assessing efficacy of a therapeutic agent for treating Alzheimer's Disease (AD) in a subject, comprising:
(1) comparing the subject's plasma or serum or whole blood levels of any one protein selected from Tables 1-4 before and after administration of the therapeutic agent to the subject;
(2) detecting a decrease in the subject's plasma or serum or whole blood level of the protein (which has a positive β value in Table 1, 2, 3, or 4) or an increase in the subject' plasma or serum or whole blood level of the protein (which has a negative β value in Table 1, 2, 3, or 4) after administration of the therapeutic agent; and
(3) determining the therapeutic agent as effective for treating AD.

38-43. (canceled)
